(19) United States
(12) Patent Application Publication
(10) Pub. No.: US 2003/0133910 A1
(43) Pub. Date: Jul. 17, 2003

(54) Wild-type ras as a cancer therapeutic agent

(76) Inventors: Ming You, Columbus, OH (US); Yian Wang, Columbus, OH (US); Zhongqui Zhang, Columbus, OH (US)

Correspondence Address: CALFEE HALTER & GRISWOLD, LLP, 800 SUPERIOR AVENUE, SUITE 1400, CLEVELAND, OH, 44114, US

(21) Appl. No.: 10/227,596

(22) Filed: Aug. 23, 2002

Related U.S. Application Data
(60) Provisional application No. 60/314,693, filed on Aug. 24, 2001.

(51) Int. Cl. A61K 48/00; C12N 15/867

(52) U.S. Cl. 424/93.2; 514/44; 435/456

(57) ABSTRACT

Methods for inhibiting proliferation of a cell, particularly a cancer cell are provided. The method comprises increasing intracellular levels of one or more wild-type ras proteins in the cell. In one aspect, the method comprises introducing and expressing a nucleic molecule that encodes a wild-type ras protein in the cell. In another aspect, intracellular levels of one more ras proteins are increased by introducing one or more wild-type ras proteins into the cell. The present invention also relates to the therapeutic use of wild-type ras proteins or nucleic acids that encode one or more wild-type ras proteins in the treatment of tumors or cancer, or prophylactically to prevent formation of tumors or cancer. Methods of characterizing or evaluating cancer in a humans or other mammals are provided. In one embodiment, the method comprises assaying for a loss of function mutation in one or more endogenous ras alleles in the genome of tumor cells obtained from the subject. In another embodiment, the method comprises assaying for a loss of function mutation in one or more endogenous ras alleles and an activating mutation in one or more ras alleles in tumor cells obtained from the subject.

This application claims priority from U.S. Provisional Application Serial No. 60/314,693 filed on Aug. 24, 2001, which is incorporated herein in its entirety.

This invention was supported, at least in part, by grant Nos. R01CA58554 and R01CA78797 from the National Institutes of Health. The Federal Government has certain rights in this invention.

### BACKGROUND

Ras genes are members of the ras supergene family of genes, present in various species, including humans and rodents. This supergene family consists of 50 or more genes that encode structurally related proteins which possess a guanine triphosphate binding site that is critical for many diverse intracellular signaling pathways. The ras subfamily of this supergene family is composed of H-ras, K-ras (also known as Kras2), N-ras, and the RAP and REL genes (Bredel and Pollack, 1999, Brain Res Rev, 29:232-49.; Beaupre and Kurzrock, 1999, J Clin Oncol, 17:1071-9.). The chromosomal locations of each of the human ras genes is known in the art.

The three ras genes (H-ras, K-ras and N-ras) comprise six or seven exons spread over between about 7 to 35 kilobase pairs of genomic DNA. Depending on the particular ras gene, there are four or five exons that containing protein-encoding regions. Within the first three of these coding exons are regions encoding the GDP/GTP binding and GTPase functions of ras proteins. The three ras genes encode p21 proteins of approximately 189 amino acids in length, which are located at the inner surface of the cell membrane when in the active GTP bound state. In particular, when a tyrosine kinase receptor is activated by binding of its ligand (e.g., a growth factor or hormone), intracellular signaling is initiated by the activation of the p21-ras protein (Bredel and Pollack, 1999, Brain Res Rev, 29:232-49.; McCormick, 1995, Curr Opin Genet Dev, 5:51-5.; McCormick, 1994, Curr Opin Genet Dev, 4:71-6.). The subsequent downstream signaling in the ras/Erk pathway activates numerous transcription factors, which regulate cellular proliferation or differentiation depending on the cell types and external stimuli.

The ras/Erk pathway is negatively regulated by several GTPase-activating proteins (GAPs). When a protein complex is formed between p21-ras and GAP, the weak intrinsic GTPase activity of the p21-ras protein is greatly enhanced (McCormick, 1995, Curr Opin Genet Dev, 5:51-5.; Ahmadian, et al., 1996, J Biol Chem, 271:16409-15.). This increased GTPase activity catalyzes GTP to GDP and returns the active GTP:p21-ras to the inactive GDP:p21-ras which terminates the downstream signaling. Non-receptor protein tyrosine kinases which are involved in interior signaling pathways may also activate p21-ras and the ras/Erk pathway (Rozakis-Adcock, et al., 1992, Nature, 360:689-92.; McCormick, 1993, Ciba Found Symp, 176:1-5; Baltensperger, et al., 1993, Science, 260:1950-2.). The regulation of the p21-ras proteins and the ras/Erk pathway is controlled by the interactions and enzymatic activities of many proteins discussed in the references cited above.

A particularly important aspect of ras genes is that they play a key role in oncogenesis. In their wild-type state, ras genes are non-oncogenic. However, various mutations of ras, so-called “activating” mutations, cause ras genes to become oncogenic in that their expression causes malignancies in animals and transformation of cultured cells. In their wild-type, non-oncogenic state, ras genes are called proto-oncogenes. Ras genes containing activating mutations are said to be oncogenes.

Activating point mutations in the ras genes have been detected in more human tumor types and at a higher frequency, 25 to 30% of all human tumors, than any other oncogene (Beaupre and Kurzrock, 1999, J Clin Oncol, 17:1071-9.; Anderson, et al., 1992, Environ Health Perspect, 98:13-24.; Bos, 1989, Cancer Res, 49:4682-9.; Rodenhuis and Slebos, 1992, Cancer Res, 52:2665s-69s.). For example, mutations are detected in 40 to 50% of colon carcinomas (Bos, et al., 1987, Nature, 327:293-7.; Forrester, et al., 1987, Nature, 327:298-303.; Burmer, et al., 1991, Environ Health Perspect, 93:27-31.); 80% of pancreatic carcinomas (Burmer, et al., 1991, Environ Health Perspect, 93:27-31.; Mariyama, et al., 1989, Jpn J Cancer Res, 80:622-6.; Shibata, et al., 1990, Cancer Res, 50:1279-83.; Pinto, et al., 1997, Acta Cytol, 41:427-34.); and 30 to 50% of lung adenocarcinomas (Rodenhuis and Slebos, 1992, Cancer Res, 52:2665s-69s.; Rodenhuis, et al., 1988, Cancer Res, 48:5738-41.; Reynolds, et al., 1991, Proc Natl Acad Sci USA, 88:1085-9.; Suzuki, et al., 1990, Oncogene, 5:1037-43.; Li, et al., 1994, Lung Cancer, 11:19-27.; Mills, et al., 1995, Cancer Res, 55:1444-7.). K-ras mutations account for >90% of the activating ras mutations observed in these tumor types. Additionally, activating ras mutations are found in hematopoietic neoplasia of myeloid origin, seminoma, breast carcinoma, glioblastoma and neuroblastoma. Ras mutations are also found in cancers of the stomach, urinary bladder, prostate, skin, thyroid, head and neck, endometrium, liver, ovary, kidney, testis, and others. Activated ras oncogenes have also been detected in spontaneously occurring and chemically-induced tumors generated in numerous rodent systems (Anderson, et al., 1992, Environ Health Perspect, 98:13-24.; Balmain and Brown, 1988, Adv Cancer Res, 51:147-82; Guerrero and Pellicer, 1987, Mutat Res, 185:293-308.). For example, K-ras oncogenes are detected in mouse lung tumors.

Not all mutations within ras are “activating” because not all mutations result in ras genes that are oncogenic. Activating mutations of ras proto-oncogenes occur predominantly, but not always, in the 12^{th}, 13^{th}, and 61^{st} codons of the genes. Activating mutations are also found in codons 59, 63, 116, 117, 119, 145 and 146. The result of these mutations is to continually up-regulate the ras/Erk signaling pathways in cells in the absence of external stimuli. Therefore, a signaling pathway which plays an important role in cell proliferation is constitutively turned on (McCormick, 1994, Curr Opin Genet Dev, 4:71-6.; Lowy and Willumsen, 1993, Annu Rev Biochem, 62:851-91). The first observed difference in biological activity between a mutant oncogenic p21 and wild-type p21 was the intrinsic GTPase activity, which was 10-times higher in wild-type as compared to the valine-12 oncogenic mutant. Later, it was realized that intrinsic GTPase activity could not be used to differentiate between some mutant forms of p21 observed in human tumors and the wild-type ras protein (Trahey and McCormick, 1987, Science, 238:542-5.). After discovery of GAP, it was shown that the main biochemical difference between oncogenic p21s with mutations in codon 12, 13, or 61 and the wild-type p21 is the ability of GAP to induce GTP hydrolysis in the active p21:GTP complex. The GAP-induced hydrolysis can be as much as 1000-times greater in the wild-type p21 than in these mutant forms of ras (Vogel, et al., 1988, Nature, 335:90-3.; Gibbs, et al., 1988, Proc Natl Acad Sci USA, 85:5026-30.). The mutant forms thus remain in the active GTP form much longer than the wild-type, and the continual transmission of a signal by the mutant forms is responsible, at least in part, for the oncogenic properties.

Activated ras oncogenes have been referred to as genetically “dominant.” A dominant genetic mutation is one that produces its phenotype even though its homologous, wild-type allele is also expressed in the same cell. The phenotype produced by the mutant gene product is said to dominate the phenotype resulting from expression of the wild-type allele of the same gene. Since activated ras alleles are oncogenic, even when wild-type alleles are also expressed, ras oncogenes have been referred to as dominant mutations (Barbacid, 1987, Annu Rev Biochem, 56:779-827; Marshall, 1991, Pa Med, 94:10.).

The characterization of ras oncogenes as dominant, however, may not be satisfactory. A gene encoding a true “dominant” mutation produces its phenotype at a normal level of gene expression (not an elevated or overexpressed level) and, as stated above, in the presence of expression of its homologous, non-mutated allele. Some studies indicate that transforming ras oncogenes are not expressed at normal levels, but instead at much higher than normal levels. In mouse NIH/3T3 cells, for example, as well as other rodent cells transformed in vitro with mutant ras alleles, the mutant ras alleles are expressed at much higher levels than in control cells (Hua, et al., 1997, Proc Natl Acad Sci USA, 94:9614-9.; You, et al., 1989, Proc Natl Acad Sci USA, 86:3070-4.; Guerrero, et al., 1984, Proc Natl Acad Sci USA, 81:202-5.; Spandidos and Wilkie, 1984, Nature, 310:469-75.). In addition, the mutant EJ H-ras1 allele, which was the first oncogenic ras allele isolated from a human tumor cell line, contains not only a 12^{th} codon mutation but also a mutation in the last intron which increases its expression at least ten-fold compared to the normal ras allele (Cohen and Levinson, 1988, Nature, 334:119-24.). Finally, Finney and Bishop (Finney and Bishop, 1993, Science, 260:1524-7.) used homologous recombination to replace one wild-type H-ras allele with a transforming mutant H-ras allele. Although both the normal and mutant H-ras allele were equally expressed, the heterozygous cells were not transformed. Spontaneously transformed cells arose from cultures of the heterozygous cells and the transformed cells had amplified the mutant allele. These particular studies indicate that the oncogenic potential of ras oncogenes may depend on high expression levels, a requirement inconsistent with a dominant mutation.

Because the oncogenic activity of activated ras oncogenes is not properly characterized as activity of a dominant mutation, there is a need for a more complete description of the transforming or oncogenic activity of activated ras oncogenes. Such a description would provide better treatments for cancers, especially those caused by activated ras oncogenes. Such a description would also provide better diagnostic and prognostic methods for cancers caused by ras oncogenes.

Existing anti-ras therapeutics take advantage of the fact that ras proteins are synthesized as inactive precursors that must be modified post-transcriptionally to be biologically active (Casey, et al., 1989, Proc Natl Acad Sci USA, 86:8323-7.; Hancock, et al., 1989, Cell, 57:1167-77.). The first step in such modification is the addition of a 15-carbon isoprenoid (i.e., farnesol) by farnesyltransferase (FTase) to the C-terminal cysteine of the CAAX motif (Hancock, et al., 1989, Cell, 57:1167-77.). Geranylgeranylation is another post-transcriptional step resulting in the addition of a 20-carbon moiety to the CAAX motif by geranylgeranyltransferase-I (GGTase-I). Existing anti-ras therapeutics exploit strategies that selectively interfere with these post-transcriptional steps (Gibbs, et al., 1993, J Biol Chem, 268:7617-20.; Tamanoi, 1993, Trends Biochem Sci, 18:349-53.; Lemer, et al., 1995, J Biol Chem, 270:26770-3.). One problem with these strategies is that inhibition of these post-transcriptional steps inhibits the activity not only of activated ras proteins, but also any existing wild-type ras proteins that have tumor-suppressor activity. Another problem is that proteins other than ras proteins are post-transcriptionally modified as described above. Therefore, inhibition of these steps are nonspecific in that proteins other than ras are likely affected. The methods described in the present application, expression of wild-type ras, are not subject to these deficiencies.

### SUMMARY OF THE INVENTION

We have found that wild-type ras genes can suppress the oncogenic or transformed phenotype of a cell (i.e., inhibiting cell proliferation), particularly when the oncogenic or transformed phenotype is caused by an activated ras gene or an activated ras signaling pathway. Therefore, the present invention provides a method for inhibiting proliferation of a cell, particularly a cancer cell. The method comprises increasing intracellular levels of one or more wild-type ras proteins in the cell. In one aspect, the method comprises introducing a nucleic molecule into the cell that encodes a wild-type ras protein. The introduced nucleic acid can be DNA, in which case the nucleic acid further comprises a transcriptional promoter which is operably linked to the sequence that encodes the wild-type ras protein. The introduced nucleic acid can also be RNA that encodes the wild-type ras protein or fragment and is capable of being translated into a polypeptide within the cell. In both cases, an increase in the intracellular levels of one or more wild-type ras proteins occurs and cell proliferation is inhibited. When the proliferating cells are within a tumor of a human or other animal, inhibition of proliferation of the cells stops or slows enlargement of the tumor, decreases the size of the tumor, or even eliminates the tumor from the body of the human.

In another aspect, intracellular levels of one more ras proteins are increased by introducing one or more wild-type ras proteins into the cell. Introduction of the wild-type ras protein into the cell may be facilitated by adding a protein transduction domain to the protein. The protein transduction domain facilitates uptake of the protein. Such internalized ras proteins inhibit proliferation of the cells that have taken up the protein. Ras proteins containing protein transduction domains can be efficiently administered to a human or other animal by routes such as injection or inhalation.

The present invention also relates to the therapeutic use of wild-type ras proteins or nucleic acids that encode one or more wild-type ras proteins in the treatment of tumors or cancer, or prophylactically to prevent formation of tumors or cancer. Human groups exist that have an above average probability of being diagnosed with particular cancers (i.e., high-risk groups) and are likely candidates for prophylactic use of wild-type ras. Such human groups may be at high risk because of exposure to particular environmental circumstances (e.g., heavy smokers), because of familial susceptibility to certain cancers (e.g., genetic inheritance of genes causing increased susceptibility), or for other reasons. Such therapeutic or prophylactic use of wild-type ras involves administration to an individual, of a therapeutically effective amount of one or more wild-type ras proteins or nucleic acid molecules encoding such proteins.

The present invention also provides a method for decreasing the activity of ERK MAP kinase in a cell by increasing the concentration of wild-type ras protein, or a growth suppressing fragment thereof, within the cell. Increasing concentrations of ras within cells is provided either by introducting and expressing a DNA molecule encoding a wild-type ras protein which is the cell, or by introducing one or more wild-type ras proteins into the cell.

The present invention also provides a method of characterizing or evaluating cancer in a human, or other mammals. In one embodiment, the method comprises assaying for a loss of function mutation in one or more endogenous ras alleles in the genome of tumor cells obtained from the subject. In another embodiment, the method comprises assaying for a loss of function mutation in one or more endogenous ras alleles and an activating mutation in one or more ras alleles in tumor cells obtained from the subject. In certain cases the loss of function mutation results in production of decreased levels of ras protein in the cancer cell. In other cases, the loss of function mutation results in production of a ras protein that has lost the growth-suppressing function possessed by a wild-type ras protein. Since the most aggressive of cancers caused by activated ras contain both an activated ras allele and a second ras allele lacking the growth-suppressing activity of wild-type ras, this method provides diagnosis of such cancers and prognosis for humans or animals that have such cancers.

The present invention also relates to a method of inducing lung tumor formation in a heterozygous ras-knockout mouse.

### BRIEF DESCRIPTION OF THE FIGURES

FIG. 1. Lung tumor bioassays using heterozygous K-ras-deficient mice. a, Experimental Design; b & d, Lung tumor multiplicity in urethane- and MNU-treated mice, respectively; c & e, Lung tumor load in urethane- and MNU-treated mice, respectively. Blank bars represent K-ras^{+/+} mice and black bars are K-ras^{+/-} mice. A/J, (A/J×129/Sv-K-ras)F₁; 129/SvlmJ, (129/SvlmJ×129/Sv-K-ras)F₁; C57BL/6J, (C57BL/6J×129/Sv-K-ras)F₁. Asterisks indicate P<0.0001 compared to wild-type animals.

FIG. 2. Lung tumors from heterozygous K-ras-deficient mice. a & b: Gross photomicrographs of lungs from (A/J×129/Sv-K-ras^{+/+})F₁ and (A/J×129/Sv-K-ras^{+/-})F₁ mice, respectively. Note the marked increase in tumor multiplicity in K-ras deficient mice compared to wild-type mice; c & d: Light photomicrographs of lung tumors (adenomas vs. carcinomas) at 4× magnification from (A/J×129/Sv-K-ras^{+/+})F₁ and (A/J×129/Sv-K-ras^{+/-})F₁ mice, respectively; e & f Lung tumors at 40× magnifications from (A/J×129/Sv-K-ras^{+/+})F₁ and (A/J×129/Sv-K-ras^{+/-})F₁ mice, respectively. Neoplastic cells in alveolar bronchiolar adenomas (e) are monomorphic while neoplastic cells in alveolar bronchiolar carcinomas (f) are pleomorphic; nuclei vary in size and shape and contain multiple prominent nucleoli.

FIG. 3. LOH on chromosome 6 in mouse lung tumors. a. A representative figure for PCR-RFLP analysis of K-ras intron 2 polymorphic region for LOH status in lung tumors from (C57BL/6J×129/Sv-K-ras^{+/+})F₁ wild-type mice. Lanes 1, A/J mouse control DNA; Lane 2, C57BL/6J mouse control DNA; Lane 3-7, lung tumors from (C57BL/6J×129/Sv-K-ras^{+/+})F₁ wild-type adenomas. b. A representative figure for LOH analysis of chloroprene-induced B6C3F1 mouse lung adenocarcinomas using K-ras codon 61 mutation (CAA→CTA) as a marker. Panel 7, B6C3F1 mouse control DNA; Panel 3, a chloroprene-induced B6C3F1 mouse lung adenocarcinoma without LOH; Panel 1,2,4-6, chloroprene-induced B6C3F1 mouse lung adenocarcinomas with LOH. c. A representative figure for the marker D6MCO12 is shown. Lanes 1, C3H/HeJ mouse control DNA; Lane 2, C57BL/6J mouse control DNA; Lane 3, B6C3F1 mouse control DNA; Lanes 4-18, chloroprene-induced B6C3F1 mouse lung adenocarcinomas.

FIG. 4. Growth suppression by wild-type K-ras in a transformed NIH/3T3 cell line (R16). a. Growth curves of the vector control vs. K-ras4B transfected R16 cells. R16 cells at the same passage level were transfected under identical conditions with 1.5-2 μg of purified plasmid DNA of either pCR3.1 vector alone or wild-type K-ras4B. G418-resistant R16 cells were counted on days 1-6. Each data point is a mean of two experiments. b & c, inhibition of colony formation by transfected wild-type K-ras4B. One thousand G418-resistant R16 cells were seeded into each of the four dishes (10-cm), and incubated in DMEM plus 10% FBS and 50 μg/ml G418 for 12 days. Cells were then fixed with 10% buffered formalin, stained with crystal violet, and visible colonies (>1.5 mm in diameter) counted. Asterisks indicate P<0.05 compared to vector transfected cells.

FIG. 5. Growth suppression by wild-type K-ras in a mouse lung tumor cell line (LM2). a. Growth curves of the vector control vs. K-ras4B transfected LM2 cells. LM2 cells of the same passage were transfected under identical conditions with 1.5-2 μg of purified plasmid DNA of either pCR3.1 vector alone or wild-type K-ras4B. G418-resistant LM2 cells were subcloned into stable clones. One of the clones is named LM2-4S-7 which expressed a relatively high level of transfected wild-type K-ras and was designated as LM2-K-ras4B H. Another clone, was named LM2-4S-4, expressed ˜3-fold lower level of transfected wild-type K-Ras and was designated as LM2-K-ras4B L. G418-resistant vector control, LM2-K-ras4B H, and LM2-K-ras4B L cells were counted on days 1-4. Each data point is a mean of two experiments. Asterisks indicate P<0.05 compared to vector transfected cells. b & c, inhibition of colony formation by transfected wild-type K-ras4B. One thousand G418-resistant vector control, LM2-K-ras4B H, and LM2-K-ras4B L cells were seeded into each of the four dishes (10-cm), and incubated in CMRL 1066 plus 10% FBS and 50 μg/ml G418 for 12 days. The cells were then fixed with 10% buffered formalin, stained with crystal violet, and visible colonies (>1.5 mm in diameter) counted. Asterisks indicate P<0.05 compared to vector transfected cells. d. RT-PCR of unique sequences carried by the transfected K-ras4B clones. Lanes 1-14: LM2-4S-1, LM2-4S-2, LM2-4S-3, LM2-K-ras4B L, LM2-K-ras4B H, LM2-4S-8 LM2-4S-9 LM2-4S-10, LM2-4S-11, LM2-4S-L1, LM2-4S-L3, LM2-4S-L4, LM2-4S-M, and vector control.

FIG. 6. Activation status of K-Ras and ERK in LM2 cells expressing wild-type K-Ras. a. Ras activation. Lysates from the LM2 cell lines were incubated with GST-RBD coupled to glutathione agarose or glurathione agarose alone (left panel). Bound K-Ras was detected by western blot with α-KRas F234 antibody (Santa Cruz Biotechnology). HEK 293 cells were transfected with expression vectors for K-RasV12 and K-RasN17, lysed and subject to the same binding assay as above (right panel). b. ERK activity. Total cell lysates were probed with anti-ERK (top panel) and anti-phospho-ERK (bottom panel) antibodies. HEK293 cells without stimulation with EGF was used as negative control and HEK293 cells stimulated with EGF was included as a positive control. The doublets in the immunoblot correspond to ERK1 (44 KD) and ERK2 (42 KD).

FIG. 7. Nude mouse tumorigenecity assays of wild-type K-ras transfected tumor cells. a & b, inhibition of nude mouse tumor development (left side—vector control; right side—K-ras4B transfected cells); c, measurement of the tumor size; d, measurement of the final nude mouse tumor weight. Asterisks indicate P<0.05 compared to vector transfected cells.

FIG. 8. A, representative autoradiograph showing LOH in non small cell lung cancer. Above each autoradiograph, microsatellite marker; arrowheads, the allele losses; N, normal DNA; T, Tumor DNA. For each sample, the left panel, lung tumor without LOH; the right panel, lung tumor with LOH. B, representative example for Kras2 gene codon 12 mutation. On the left, lung tumor without Kras2 mutation; on the right, lung tumor with Kras2 gene 12^{th} codon mutation (GGT→TGT transition).

FIG. 9. LOH map of chromosome 12 in non small cell lung cancer. Fourteen samples that exhibited LOH at one or more loci are presented. On the left, 12 samples also retained activated Kras2 allele. On the right, 2 samples did not contain Kras2 mutation. The Genetic map of chromosome 12 was derived from NCBI Human Genome Resources. L, large cell carcinoma; A, adenocarcinoma.

### DETAILED DESCRIPTION OF THE INVENTION

Definitions

Herein, “wild-type”, refers to the nucleotide sequence of a gene, or the amino acid sequence of the protein encoded by that gene, that is the sequence found in the majority of organisms or cells that contain the specific gene. If a gene or protein is not wild-type it is mutant. A mutant gene has a nucleotide sequence that is different from the sequence of the wild-type gene. A mutant protein has an amino acid sequence that is different from the sequence of the wild-type protein.

Herein, wild-type ras proteins have an activity called “growth suppressing” activity. After expression of wild-type ras activity, or additional wild-type ras activity, within a cell, the growth suppressing activity is measured as a decrease or inhibition of one or more growth or proliferation parameters of the cell as compared to cells in which the wild-type ras or additional wild-type ras activity was not expressed. Many different measurements made on these cells are indicative growth suppressing activity. For example, decrease in cell cycle time, decrease in cell division time and decrease in G1 cell cycle phase duration are indicators of growth suppressing activity of wild-type ras. If the cells in which wild-type ras activity is expressed are tumor, cancer or transformed cells, measurement of properties such as decrease in efficiency of colony formation in soft agar or decrease in focus formation are indicators of growth suppressing activity of wild-type ras as compared to controls. If the cells in which wild-type ras activity is expressed comprise or are part of a tumor within a human or animal, measurement of properties such as decreased tumor size, decreased tumor growth rate or slower rate of metastasis are indicators of growth suppressing activity of wild-type ras.

An important of this invention is that the wild-type ras gene that is expressed in the cells inhibits or suppresses cell proliferation. Inhibition or suppression of cell proliferation are terms well known to those skilled in the art and refer to slowing or stopping of cell division such that cells do not increase in number. The magnitude of such slowing of cell growth can be variable. Herein, any alteration of the growth of cells that comprise tumors falls within the scope of this application. A variety of measurements of characteristics of cells indicative of slowing or stopping of cell growth are described in the Examples of this application. Additional aspects of cell growth suppression by wild-type ras of cells comprising a tumor that is within the scope of this application are apoptosis (programmed cell death) and induction of cell differentiation.

Herein “inactivating mutations” or “loss of function mutations” refer to mutations in or around ras genes that eliminate or decrease the growth suppressing activity of ras proteins encoded by such genes. One example of this is one or more substitutions, deletions, insertions or duplications within the coding region of the gene that does not result in a decrease in the amount of ras protein expressed by the gene, but on a per molecule basis, the ras protein containing the mutation has quantitatively less growth suppressing activity than a ras protein not containing an inactivating mutation. This type of inactivating mutation is likely to occur within one or more of the first three coding exons of ras gene.

Another example of an inactivating mutation is a mutation in, near or affecting a ras gene such that no or a decreased amount of ras protein is expressed from that gene. Such mutations, for example, can decrease transcriptional activity of a ras promoter, cause aberrant or decreased splicing of transcripts from the gene, or cause decreased half-life of ras mRNA or protein within the cells. In this case, although the ras protein produced may have quantitatively the same growth suppressing activity as a wild-type ras protein on a per molecule basis, the cells containing such a mutation have decreased amounts of wild-type ras protein and, therefore, decreased amounts of ras growth suppressing activity.

Other mutations result in deletion of an entire ras allele, or deletion of enough of a ras allele so that no protein is produced from that DNA sequence. Other types of mutations include internal duplications within a ras gene sequence, one type being tandem duplications.

It should also be recognized that decreases in the amount of wild-type ras growth suppressing activity within a cell can also result from an increase or change in the methylation of cytosine residues in the DNA, within or near a particular ras gene. Such epigenetic changes in the DNA of a cell result in hypermethylation, are well known in the art and are generally known to result in decreased levels of transcription from a gene.

It should be recognized that the growth-suppressing activity of wild-type ras correlates with a decrease in the activity of ERK MAP kinase in a cell. ERK MAP kinase is downstream in the ras signaling pathway, which is well known in the art. Increasing concentrations of wild-type ras protein within cells results in decreased activity of ERK MAP kinase (See Example 2).

The present invention also provides a method for decreasing the activity of ERK MAP kinase in a cell by increasing the concentration of wild-type ras protein within the cell.

Herein “activating mutations” or “gain of function mutations” refer to mutations, generally within the coding sequence of ras genes, that result in production of ras proteins that have transforming, tumorigenic or carcinogenic activity. Biochemically, ras proteins containing activating mutations, also called activated ras proteins, may have reduced GTPase activity, so that the ras proteins are locked in the active GTP-bound state. Activated ras proteins may also have decreased nucleotide binding affinity and, therefore, GDP bound by ras is exchanged for GTP at an increased rate. It may also be that GAP proteins have a decreased ability to cause the conversion of GTP-bound ras to GDP-bound ras. All of these cases result in accumulation of GTP-bound ras, which is the ras which causes signal transduction. Activating mutations are known to be found in codons 12, 13, 59, 61, 63, 116, 117, 119, 145 and 146 of ras genes. Such mutations occur at or near the guanine nucleotide binding sites of the protein. Not all mutations within these codons cause activation of ras to the same degree. Likewise, not all mutations within these codons are activating. Some mutations in one or more of these codons may be loss of function mutations. Activating mutations are detected by a number of techniques, some of which are described in U.S. Pat. No. 5,591,582 by Bos, et al., the information within which is hereby incorporated by reference.

Herein, “introduction” of a DNA molecule into a cell refers to a variety of methods known in the art to get DNA molecules into cells. One such method, whereby isolated DNA is introduced into cells, is know as transfection. Such transfection is commonly performed using various treatments of the cells or DNA which facilitate uptake of the DNA by the cell. For example, cells can be treated chemically to make them permeable to DNA. DNA can also be treated, for example by incorporating the DNA into liposomes that cells can internalize.

Nucleic acids (DNA or RNA) can also be introduced into cells using viruses. For example, a cDNA encoding a wild-type ras protein that is to be introduced into cells is cloned into a viral genomes. Infection of cells with such viruses results in introduction of the viral genome into the cell. Since the cloned gene is part of the viral genome, it is introduced into the cell along with the viral genome. Such viral “vectors” can have DNA or RNA genomes. Numerous such viral vectors are well known to those skilled in the art. Viral vectors that have genes, wild-type ras for example, cloned into their genomes can be referred to as “recombinant” viruses.

Herein, “oncogenic”, “transformed” and “neoplastic” refer to cells that possess characteristics (i.e., phenotypes) of cancer or malignant cells. A variety of such characteristics are well known to those skilled in the art. Oncogenic cells, when injected into animals, for example, form tumors whereas non-oncogenic cells do not form tumors after injection into an animal. Herein, so-called “oncogenes” are genes that, after introduction into cells and expression therein, cause non-cancerous cells to become cancerous. Herein, one such oncogene is a ras gene containing an activating mutation. Such a ras gene can be called an “activated” ras gene. “Anti-oncogenes” or “tumor-suppressor” genes are genes that, after introduction into cells and expression therein, cause cancerous cells to become non-cancerous. Herein, one such tumor-suppressor gene is a wild-type ras gene.

“Neoplasia” means the process resulting in the formation and growth of an abnormal tissue that grows by cellular proliferation more rapidly than normal, and continues to grow after the stimuli that initiated the new growth cease.

Tumor Incidence in Mice with Different Copy Numbers of Wild-Type ras

These studies were designed to determine the effect of wild-type K-ras in lung tumorigenesis. Mice lacking both K-ras alleles are not viable and die between 12 and 14 days of gestation due to fetal liver defects and anemia (Johnson, et al., 1997, Genes Dev, 11:2468-81.), therefore, they could not be tested. However, heterozygous K-ras deficient mice are an excellent alternative to assess the role of wild-type K-ras in lung tumorigenesis since nearly 100% of the lung tumors from inbred strains of mice exhibit an activated K-ras allele (You, et al., 1989, Proc Natl Acad Sci USA, 86:3070-4.). These studies were performed as described below.

Animals and Generation of F1 Heterozygotes

Six-week-old A/J, 129/SvlmJ, and C57BL/6J female mice were obtained from the Jackson Laboratories (Bar Harbor, Me.). The 129/Sv-K-ras^{+/-} (K-ras “knockout”) mice were developed as reported previously (Johnson, et al., 1997, Genes Dev, 11:2468-81.). The knockout mice contained an insertion mutation of a DNA construct containing a neo gene into the K-ras gene (Johnson, et al., 1997, Genes Dev, 11:2468-81.). Animals were housed in plastic cages with hardwood bedding and dust covers, in a HEPA filtered, environmentally controlled room (24±1° C., 12/12 hr light/dark cycle). Animals ate Rodent Lab Chow (#5001; Purina) and drank water ad libitum. Following a 7-day quarantine, the animals were paired to develop breeding colonies for production of (A/J×129/Sv-K-ras^{+/-})F₁, (129/SvlmJ×129/Sv-K-ras^{+/-})F₁, and (C57BL/6J×129/Sv-K-ras^{+/-})F₁ mice. Body weights of all the animals were monitored monthly for the duration of the studies.

Genotyping of Mice

Tail tissue clippings were taken from each (A/J×129/Sv-K-ras^{+/-})F₁, (129/SvlmJ×129/Sv-K-ras^{+/-})F₁, and (C57BL/6J×129/Sv-K-ras^{+/-})F₁ mouse, homogenized and incubated overnight at 37° C. in lysis solution (pronase 0.4 mg/ml, 10% sodium dodecylsulfate (w/v), 10 mM Tris, 400 mM NaCl, and 2 mM EDTA), followed by phenol-chloroform extraction and precipitation with ice-cold alcohol. DNA isolated from clipped tails of each mouse was genotyped for the presence of the K-ras targeted mutation using polymerase chain reaction (PCR). Briefly, a pair of PCR primers (oIMR013: 5′-CTTGGGTGGAGAGGCTATTC-3′, oIMR014: 5′-AGGTGAGATGACAGGAGATC-3′) was used to amplify a 280 bp product from the neo insert. Another pair of PCR primers (oIMR015: 5′-CAAATGTTGCTTGTCTGGTG-3′ TCR Cd-1, oIMR016: 5′- GTCAGTCGAGTGCACAGTTT-3′) was used to amplify a 150 bp product as an internal standard. DNA having both wild-type K-ras alleles [K-ras (+/+)] displayed only a single 150 bp fragment, whereas DNA with a wild-type K-ras allele and targeted mutation (+/−) allele showed 150 bp and 280 bp bands after PCR. This screening was repeated at least once for confirmation. The heterozygote male 129/Sv-K-ras^{+/-} mice mated with A/J and C57BL/6J females. Fifty percent of the offspring had a K-ras targeted mutation (tm1), and the remaining 50% had only the wild-type K-ras allele.

Lung Tumor Formation in K-ras^{+/-} Mice

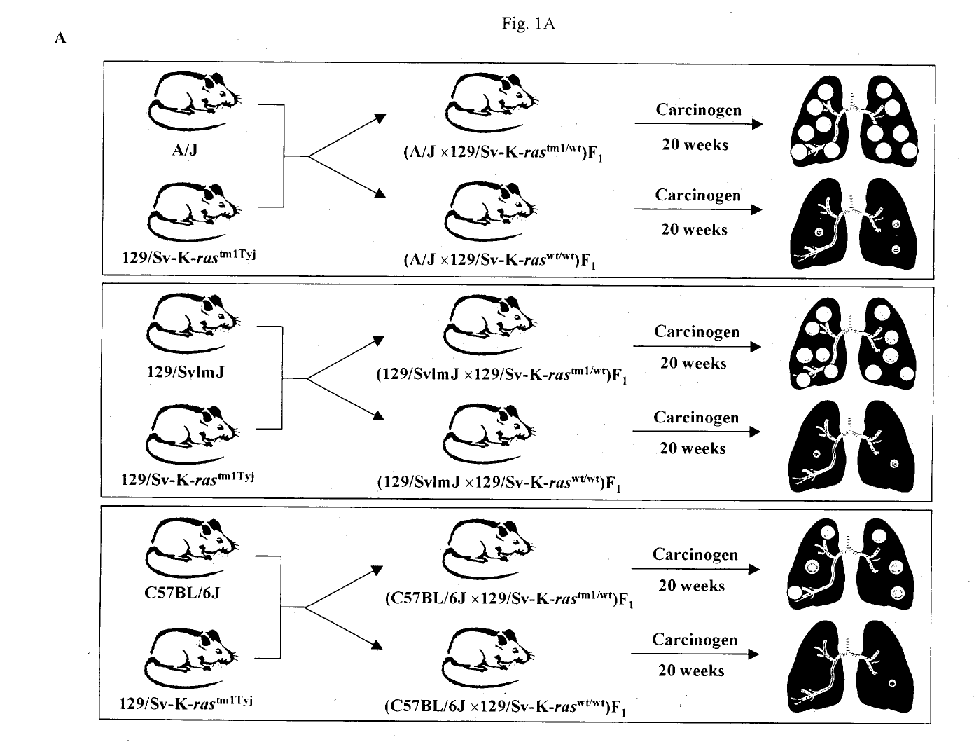

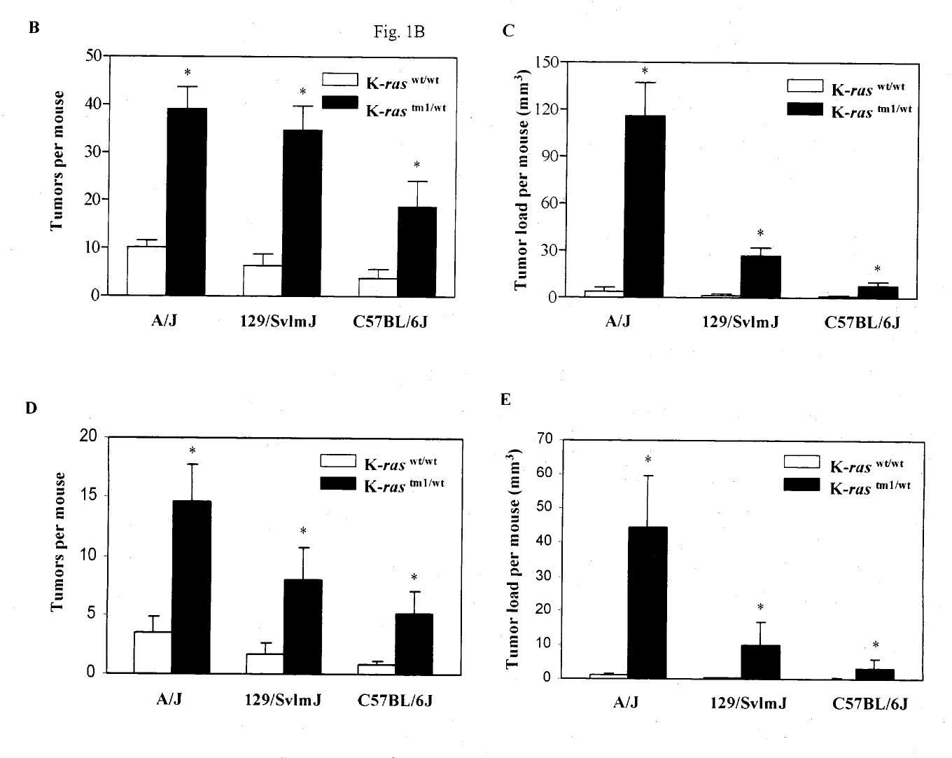

The experimental design for both urethane- and MNU-induced lung tumorigenesis studies is shown in FIG. 1a. Six-week-old (A/J×129/Sv-K-ras^{+/-})F₁, (129/SvlmJ×129/Sv-K-ras^{+/-})F₁, and (C57BL/6J×129/Sv-K-ras+/−)F₁ hybrid mice were randomized into twelve groups according to the K-ras genotypes and the types of carcinogen treatments. Urethane (ethyl carbamate) (>99% pure) and N-methylnitrosourea (MNU) (99% pure) were obtained from Sigma Chemical Co. (St. Louis, Mo.). These chemical carcinogens were prepared immediately before use in bioassays. Both urethane and MNU were dissolved in normal saline. For urethane treatment, 100 male mice were used in the bioassay including 17 (A/J×129/Sv-K-ras^{+/+})F₁ mice, 12 (A/J×129/Sv-K-ras^{+/-})F₁ mice, 15 (129/SvlmJ×129/Sv-K-ras^{+/+})F₁ mice, 19 (129/SvlmJ×129/Sv-K-ras^{+/-})F₁ mice, 19 (C57BL/6J×129/Sv-K-ras^{+/+})F₁ mice, and 18 (C57BL/6J×129/Sv-K-ras^{+/-})F₁ mice. All animals were given a single intraperitoneal (i.p.) injection of urethane (1 mg/g body weight) in 0.2 ml phosphate-buffered saline.

For MNU treatment groups, 83 male mice were used in the bioassay including 14 (A/J×129/Sv-K-ras^{+/+})F₁ mice, 13 (A/J×129/Sv-K-ras^{+/-})F₁ mice, 14 (129/SvlmJ×129/Sv-K-ras^{+/+})F₁ mice, 15 (129/SvlmJ×129/Sv-K-ras^{+/-})F₁ mice, 13 (C57BL/6J×129/Sv-K-ras^{+/+})F₁ mice, and 14 (C57BL/6J×129/Sv-K-ras^{+/-})F₁ mice. All animals were given a single i.p. injection of MNU (50 mg/kg body weight) in 0.2 ml phosphate-buffered saline.

Since lung tumor development in mice is highly dependent upon different genetic backgrounds, three different F₁ mice were used to avoid possible strain-specific effects. Eighteen weeks after treatment with urethane and twenty weeks after treatment with MNU, animals from all twelve groups were euthanized by CO₂ asphyxiation. A portion of lung tumors and normal tissue were removed and flash frozen in liquid nitrogen. A representative portion of each tumor was fixed in Tellyesniczky's solution for histopathological examination. Each lung was examined with a dissecting microscope to obtain the tumor count and size. Tumor volumes were determined by measuring the three-dimensional size of each tumor and by using the average of the three measurements as the diameter. The radius (diameter/2) was determined and total tumor volume calculated by: Volume=(4/3)πr³ (r-radius). One-way ANOVA was used to determine the difference in the number of pulmonary tumors per mouse between control and treated groups. Two-way ANOVA was used, to determine the difference in both the number and the size of lung tumors between control and treated groups.

As shown in FIG. 1a, six-week-old (A/J×129/Sv-K-ras^{+/-})F₁, (129/SvlmJ×129/Sv-K-ras^{+/-})F₁, or (C57BL/6J×129/Sv-K-ras^{+/-})F₁ mice, either wild-type or heterozygous K-ras knockout, were given a single intraperitoneal injection of urethane at a dose of 1 mg/g body weight. At eighteen weeks after treatment with urethane, all three groups of heterozygous K-ras deficient mice had developed significantly more and larger lung tumors than their homozygous wild-type counterparts. As shown in FIG. 1, treatment of (A/J×129/Sv-K-ras^{+/-})F₁ heterozygous K-ras-deficient mice with urethane produced four times as many tumors per lung as did treatment of wild-type (A/J×129/Sv-K-ras^{+/+})F₁ mice with the same carcinogen. Similarly, urethane treatment of (129/SvlmJ×129/Sv-K-ras^{+/-})F₁ and (C57BL/6J×129/Sv-K-ras^{+/-})F₁ heterozygous K-ras-deficient mice resulted in 4-5 times as many tumors per lung as did treatment of their respective wild-type littermates (FIG. 1b). There were even more striking effects on total lung tumor volume: over a 30-fold, 19-fold, and 8-fold volume increase in heterozygous K-ras-deficient mice with an A/J×129/SvJ background, a 129/SvlmJ×129/Sv background, and a C57BL/6J×129/Sv mouse background, respectively (FIG. 1c).

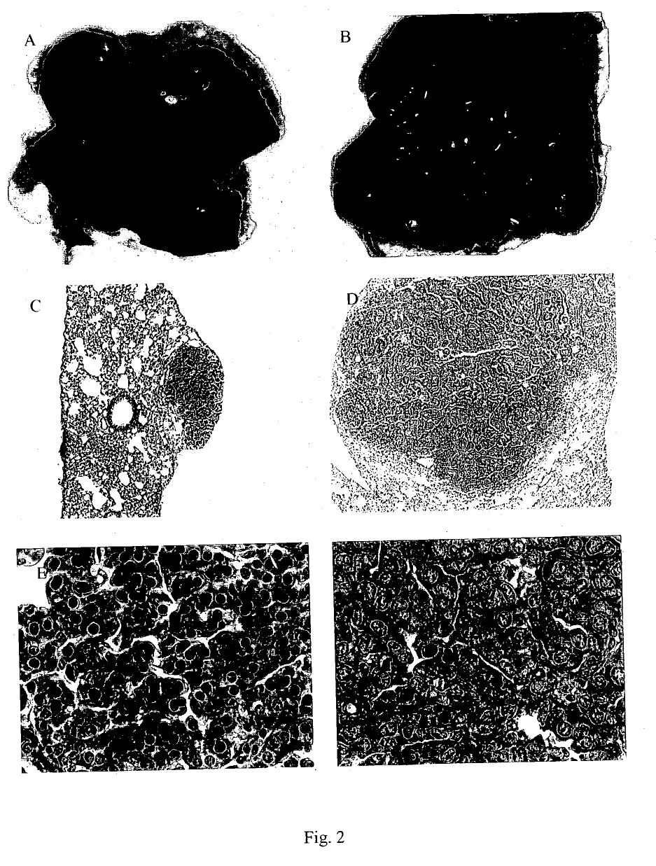

Most of the large lung tumors (>60%) from the heterozygous K-ras-deficient mice were adenocarcinomas (FIGS. 2b, d, & f). In contrast, tumors from wild-type mice treated with either urethane or N-methyl-N-nitrosourea (MNU) were all small lung adenomas (FIG. 2a, c, & e). As shown in FIGS. 2c & e, adenomas were characterized by a monomorphic growth pattern and were generally comprised of well-differentiated cells. Approximately 60% of the lung tumors from the heterozygous K-ras-deficient mice were adenocarcinomas (FIGS. 2d & f). Mouse lung adenocarcinomas were composed of cells with varying degrees of differentiation (FIG. 2f). These tumors exhibited a complete loss of normal alveolar architecture, increased nuclear/cytoplasmic ratio, nuclear crowding and cytologic atypia, heterogeneity of growth patterns, and invasion into adjacent bronchioles or vessels. These results suggest that loss of wild-type K-ras contribute significantly to the development of undifferentiated malignant lung tumors.

These observations were unexpected and indicated that the wild-type allele of K-ras acts as a tumor suppressor during chemically induced lung carcinogenesis. A second lung tumor bioassay using MNU as the carcinogen was conducted. MNU is a direct-acting alkylating agent that does not require metabolic activation, while urethane is a procarcinogen that requires metabolic activation. (A/J×129/Sv-K-ras^{+/-})F₁, (129/SvlmJ×129/Sv-K-ras^{+/-})F₁, or (C57BL/6J×129/Sv-K-ras^{+/-})F₁ mice were treated with MNU using a protocol similar to that described for urethane. As seen in FIGS. 1d & e, treatment of K-ras deficient mice with MNU produced a 4- to 6-fold increase in tumor multiplicity and a 32- to 50-fold increase in tumor volume in all three mouse backgrounds.

K-ras Activating Mutations in Lung Tumors

Lung tumors in the mice described above were analyzed for K-ras activating mutations. DNA was isolated from lung tumors using the TRIzol reagent (Gibco BRL) and was used as a template for PCR reactions and the DNA of the PCR products was then sequenced. The sequences of PCR primers for K-ras exons 1 and 2 were described previously (You, et al., 1989, Proc Natl Acad Sci USA, 86:3070-4.). PCR reactions were also carried out and direct sequencing of PCR products was performed as described previously (You, et al., 1989, Proc Natl Acad Sci USA, 86:3070-4.).

As shown in Table 1, sequence analysis performed on the first exon and the second exon of K-ras revealed that all urethane-induced tumors analyzed from K-ras wild-type mice contained an AT to TA transversion at the second position of codon 61. A similar frequency and type of K-ras mutation was observed in lung tumors from heterozygous K-ras-deficient mice treated with urethane. Similarly, all MNU-induced lung tumors from either wild-type mice or heterozygous K-ras-deficient mice contained a GC to AT transition at the second position of codon 12 in K-ras (Table 1).

TABLE 1
Activating mutations in the K-ras gene detected in lung tumors

| | Treat- | | Codon 12 | Codon 61 |
|---|---|---|---|---|
| Animals | ment | Incidence | (GGT→GAT) | (CAA→22CTA) |
| (A/J × 129/Sv-K-ras^{+/+})F₁ | urethane | 10/10 | 0 | 10 |
| (A/J × l29/Sv-K-ras^{+/-})F₁ | urethane | 10/10 | 0 | 10 |
| (129/SvlmJ × 129/Sv-K-ras^{+/+})F₁ | urethane | 9/9 | 0 | 9 |
| (129/SvlmJ × 129/Sv-K-ras^{+/-})F₁ | urethane | 9/9 | 0 | 9 |
| (C57BL/6J × 129/Sv-K-ras^{+/+})F₁ | urethane | 10/10 | 0 | 10 |
| (C57BL/6J × 129/Sv-K-ras^{+/-})F₁ | urethane | 9/9 | 0 | 9 |
| (A/J × 129/Sv-K-ras^{+/+})F₁ | MNU | 5/5 | 5 | 0 |
| (A/J × 129/Sv-K-ras^{+/-})F₁ | MNU | 5/5 | 5 | 0 |
| (129/SvlmJ × 129/Sv-K-ras^{+/+})F₁ | MNU | 5/5 | 5 | 0 |
| (129/SvlmJ × 129/Sv-K-ras^{+/-})F₁ | MNU | 5/5 | 5 | 0 |
| (C57BL/6J × 129/Sv-K-ras^{+/+})F₁ | MNU | 5/5 | 5 | 0 |
| (C57BL/6J × 129/Sv-K-ras^{+/-})F₁ | MNU | 3/3 | 3 | 0 |

LOH of the Distal Chromosome 6 in Mouse Lung Tumors

In this study, applicants further examined the relationship between K-ras activating mutations and loss of markers in the region of K-ras (loss of heterozygosity or LOH), indicating the loss of the other, wild-type allele of K-ras in tumors.

The National Toxicology Program (NTP) provided two sets of mouse lung tumors from the B6C3F1 mice for K-ras mutation and LOH analyses. The first set was from mice exposed to 0, 1, 2, or 4 mg/m³ of V2PO5 by inhalation for 2 y. The second set was from mice treated with chloroprene. Lung tumor multiplicity data and K-ras mutation analysis of chloroprene-induced tumors were reported (Sills, et al., 1999, Carcinogenesis, 20:657-62.; 1996, NTP Technical Report 467, NIH Publication no. 96-3957, NIEHS, NIH, Research Triangle Park, N.C.). Allelotypic analyses were performed at marker D6MIT14 with 42 mouse lung tumors in the V2PO5 study (2 from untreated mice and 40 from V2PO5 exposed mice). D6MIT14 was obtained from Research Genetics, Inc. (Huntsville, Ala.). Standard PCR reactions were performed and allelic losses were scored when a visible difference could be observed. Chloroprene-induced lung tumors were fixed in formalin and paraffin embedded; therefore, it was not possible to obtain satisfactory PCR results with the D6MIT14 marker. In order to assess LOH near K-ras, a marker D6MCO12 (primer sequences: D6MCO12-1F 5′GATGTCAAACGTGAGAGTGTC3′, SEQ ID NO. ______ and D6MCO12-1R 5′GCGCTGACTCGCTTCTTCCAT3′, SEQ ID NO. ______) that is polymorphic between the C57BL/6 and C3H mice was used, and single strand conformation polymorphism analysis technique. Results were further confirmed using K-ras codon 61 mutation (CAA→CTA) as a marker.

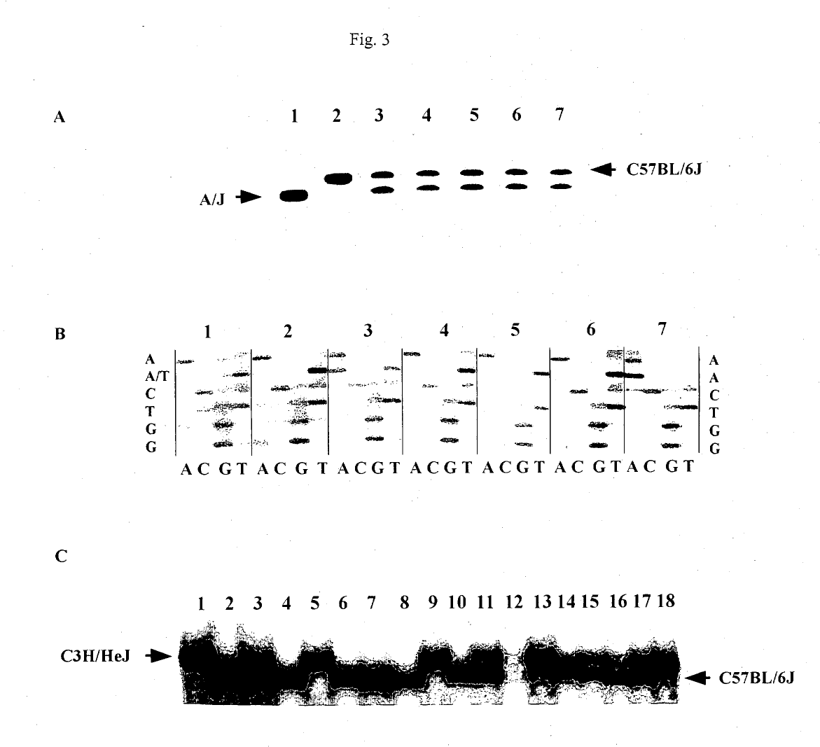

As summarized in Table 2, 40 vanadium pentoxide (V2PO5)-induced lung adenocarcinomas were examined for K-ras activation, and 29 (73%) had mutations. These tumors were also typed for LOH in the region of K-ras at marker D6MIT14, and 18 of these exhibited allelic loss (Table 2). Most frequently K-ras mutations found in V2PO5-induced tumors were either a GA to AT transition or a GA to TA transversion in the second base of codon 12 (data not shown). D6MIT14 is mapped to within 0.5-cM of K-ras (Manenti, et al., 1999, Genome Res, 9:639-46.). Allelic losses at D6MIT14 correlated closely with K-ras mutations in the tumors, with 16 of 18 (89%) tumors with allelic loss also having a K-ras mutation. Using K-ras codon 61 mutation (CAA→CTA) and D6MCO12, which is approximately 5 cM proximal to K-ras, applicants examined the extent of LOH in these samples. As shown in FIGS. 3b & c and Table 2, 16 of 19 (84%) tumors with allelic loss also having a K-ras mutation, indicating a close correlation between allelic loss at these markers and K-ras activation. These results also indicate that the lower incidence and smaller size of lung tumors in the K-ras wild-type mice is a consequence of the maintenance of the wild-type K-ras allele. PCR-RFLP analysis was performed on 18 urethane-treated lung tumors with 6 from each of (A/J×129/Sv-K-ras^{+/+})F₁, (129/SvlmJ×129/Sv-K-ras^{+/+})F₁, or (C57BL/6J×129/Sv-K-ras^{+/+})F₁ mice. None of 18 lung tumors from wt/wt mice contained an allelic loss. Representative results for analyzing LOH status in lung tumors from (C57BL/6J×129/Sv-K-ras^{+/+})F₁ mice is shown in FIG. 3a.

TABLE 2
LOH on distal chromosome 6 associated with K-ras activation in mouse lung tumors

| | | | | Frequency | K-ras |
|---|---|---|---|---|---|
| | | | Frequency | of LOH on | mutations in |
| | | | of K-ras | chromosome | tumors with |
| Strain | Treatment | Tumor type | activation | 6 | allelic loss |
| B6C3F1 | none^{a} | Adenoma | 2/14 (14%) | 3/14 (21%) | 2/3 (67%) |
| B6C3F1 | None | adenocarcinoma | 19/58 (33%) | 8/58 (14%) | 8/8 (100%) |
| B6C3F1 | MeCl^{a} | adenocarcinoma | 10/49 (20%) | 7/49 (15%) | 7/7 (100%) |
| B6C3F1 | V2PO5 | adenocarcinoma | 29/40 (73%) | 18/40 (45%) | 16/18 (89%) |
| B6C3F1 | Chloroprene | Adenoma | 13/16 (81%)^{b} | 13/16 (81%) | 12/13 (92%) |
| B6C3F1 | Chloroprene | adenocarcinoma | 6/9 (67%)^{b} | 6/9 (67%) | 4/6 (67%) |

^{a}LOH and K-ras mutation data are from Hegi et al. (Hegi, et al., 1994, Cancer Res, 54:6257-64.).
^{b}K-ras mutation data are from Sills et al. (Sills, et al., 1999, Carcinogenesis, 20:657-62.).

Diagnostic and Prognostic Assays

The present invention provides methods of characterizing tumors or cancers in a human or animal. In one aspect, the methods involve analysis of one or more alleles of one or more ras genes in a sample from a tumor or cancer for the purpose of detecting mutations in or near the ras genes. In one embodiment, the alleles for a specific H-ras, K-ras and N-ras are analyzed. In other embodirhents, the alleles for multiple of the H-, K-, or N-ras genes are analyzed. The analysis typically involves some form of genotyping of the DNA from the genome of tumor or cancer cells and can be done in a variety of ways. Preferably, the analysis involves some type of DNA sequence determination of the ras genes. Genotyping that does not involve sequence analysis may fail to detect mutations of the type where single base pairs within a gene have been changed.

In one type of analysis, DNA isolated from the tumor or cancer is analyzed by polymerase chain reaction (PCR). Regions of the ras gene, or surrounding areas, are chosen and PCR primers are made that hybridize with the genome DNA in the region. Such primers can be made to any known sequence within the ras gene or to regions surrounding the ras gene where the genomic sequence is known. One such set of regions surrounding the ras gene that can be used are polymorphic microsatellite markers, whose sequences and locations throughout the human, and some animal genomes, are known in the art. The primers are used in a PCR reaction to amplify the region of the genome that contains the ras gene of interest. A single PCR reaction may be used to amplify the entire genomic region containing the ras gene. Alternatively, multiple PCR reactions, each amplifying a different region of the ras gene of interest may be used. Preferably, PCR reactions are used such that the entire coding region of the ras gene of interest is amplified. In addition, genomic regions within introns and surrounding the ras gene of interest may also be amplified.

Analysis of the PCR products is then performed. Analysis of the PCR products can be of various types. The type of analysis performed normally depends on the type of mutation one performing the analysis is attempting to detect. For example, analysis of the size of a particular PCR product from the tumor or cancer cell genome as compared to the size of the same PCR product using DNA from a control cell (i.e., one known to have wild-type ras genes), can detect insertions or deletions of DNA in that area of the genome. It is well known in the art, that if there is an insertion of DNA in the area of a genome between the regions where two PCR primers are used to amplify the genome, the resulting PCR product is larger in size compared to the size of the same PCR product obtained using DNA from a genome where no insertion has occurred. Likewise, a deletion of DNA in the genome between two PCR primers results in a PCR product that is smaller in size compared to a control PCR product obtained using DNA from a genome not containing a deletion. Such analyses detect relatively large changes (e.g., minimum of 10% change) in size of a PCR product as compared to the product from a wild-type genome. Normally, size determination of PCR products is performed by comparing the relative sizes of two or more PCR products. For example, the size of a PCR product from a genome where a ras mutation is suspected is compared to the size of the same PCR product from a genome where ras mutations are known not to be present. Relative sizes are easily compared using migration of PCR products in an electric field, as occurs in gel electrophoresis. Agarose gel electrophoresis is often used for this purpose.

Another method for analyzing PCR products is through determination of the nucleotide sequence of all or part of the PCR product. This method of analysis detects changes in relative size of PCR products that are less than 10%. This method also detects changes in the DNA sequence that do not result in relative size changes. For example, determination of the sequence and comparison of the sequence of the same PCR product obtained from amplification of DNA from two different cells can detect single or multiple nucleotide base changes, substitutions of regions of DNA, and the like. Methods for DNA sequence determination and for DNA sequence determination of PCR products are well known in the art of molecular biology. The chain termination method of sequencing is often used. DNA sequencing is often performed by automated sequencing machines.

In another type of analysis, RNA, preferably mRNA isolated from the tumor or cancer cells is used as a template to make DNA in a reverse transcription reaction. The reverse transcribed DNA is then used as a template in PCR reactions using PCR primers with sequences known to be within the mRNA of the ras gene that is being tested. Various mRNA primers can be chosen, as described above in order to amplify the entire length of the mRNA sequence of the particular ras gene. This can be done using a single PCR reaction, or multiple PCR reactions as described above. Analysis of the PCR products is then performed much as already described. In one type of analysis, the presence of absence of a PCR product, or a change in its size as compared to controls is indicative of large changes, such as large insertions or deletions within the ras genome regions. Again, such analysis is commonly performed using gel electrophoresis of the PCR products. In another type of analysis, the DNA sequence of the PCR products is determined, using methods well known in the art.

Other methods, well known in the art, can also be used to assay for presence of ras genes, transcripts, or changes in either as compared to wild-type. Some of these methods include Southern blotting, Northern blotting, RNase protection assays, S1 nuclease assays and the like.

In addition to methods for analyzing ras DNA and RNA for mutations, it is also possible to assay ras proteins themselves, if present, for changes. For example, a variety of antibodies are well known in the art that bind specifically to different ras proteins (H-ras, K-ras and N-ras), dependent on the presence of specific mutations within the gene encoding that particular ras protein. For example, antibodies are available that bind to ras proteins only if there is an activating mutation in a specific codon of the gene encoding the protein. Other antibodies bind to the ras proteins only if the protein contains no mutations, or at least none of the well-characterized activating mutations. The basis for function of such differentially-binding antibodies generally is the conformation of the particular ras protein. For example, mutations may cause changes in the conformation of a ras protein such that it either will or won't be bound by a specific antibody. Many of these types of antibodies are available commercially (e.g., Oncogene Science). Therefore, through the use of antibodies, it can be determined whether specific mutant ras proteins are present in tumor or cancer cells. It can also be determined, whether specific mutations are present.

Such antibodies are used to analyze ras proteins using a variety of assays that are well known in the art. For example, immunoprecipitation, Western blotting and immunohistochemistry are commonly used methods. Other assays using antibodies are known in the art and can be used. Certain antibodies may give good results in one or more of such assays.

As described, hypermethylation of ras genes can result in decreased expression of wild-type ras activity in a cells. There are a variety of methods well known in the art for detecting hypermethylation of a particular gene in cells. In one method, a pair of restriction endonucleases are used that recognize the same nucleotide sequence in the DNA, but cleave the sequence differentially depending on whether particular nucleotides within the sequence are methylated. The cleaved DNA is then analyzed by Southern blotting, using a probe that spans one or more of the restriction endonuclease sites. Changes in methylation of the site in genome DNA from the tumor or cancer cells as compared to control cells is detected by differences in the banding pattern of DNA fragments on the Southern blot. Other methods for detecting hypermethylation include bisulfite treatment of the genome DNA to change the methylated cytosines therein to a different nucleotide base. The changed base is then detected using various techniques, methylation-sensitive PCR being one of these techniques. Other methods involving various DNA sequencing techniques. One such technique is genomic sequencing. Other methods are known and can be used.

The results of such assays, to characterize the ras genes, RNA or protein in tumor or cancer cells, can be used diagnostically or prognostically. Since wild-type ras has growth suppression activity, the presence of this activity is desirable for a patient on whom the assays described above are used. Generally, the least desirable situation for the patient is one where no wild-type ras growth suppression activity is present in the cells from the tumor or cancer. A more desirable situation, and one with a better prognosis, is generally one where there is wild-type ras growth suppression activity in cells of the tumor or cancer.

Wild-Type ras Genes as Therapeutic or Prophylactic Agents

The present invention also provides methods for inhibiting or suppressing growth of cells by causing increased expression in cells of wild-type ras proteins that have growth suppression activity. Increased expression of this activity can be caused by a variety of methods. Such methods normally involve introduction into the cells of a polynucleotide sequence encoding the wild-type ras protein. DNA or RNA can be introduced into such cells. It is also possible to introduce ras proteins into the cells. It is also possible, in some instances, to administer agents that cause increased expression of wild-type ras activity from genes already in the cells.

It should be appreciated that these methods are used therapeutically, in the case where a subject has a tumor or cancer that can be treated with wild-type ras. The methods can also be used to prevent the formation of a tumor or cancer in individuals likely to form these. The methods of causing increased expression of ras are preferably used in the case where an individual with a tumor or cancer has decreased or no wild-type activity in the cells of the tumor or cancer. However, the present methods can also be used in instances where there is wild-type ras activity, but the well-known ras signaling pathway is still activated in such a way that it contributes to formation and growth of the tumor or cancer. It is also possible to use the present methods in cases where genes other that ras or pathways other than the ras signaling pathway contribute to the tumor or cancer in the individual.

In these methods, “wild-type ras protein” refers collectively to any wild-type protein that is encoded by a member of the ras supergene family described earlier. More preferably, the wild-type ras proteins used are encoded members of the ras subfamily of the supergene family (i.e., H-ras, K-ras, N-ras). Most preferably, the wild-type ras protein is the K-ras protein. Preferably, the genes and proteins used in the present methods are of human origin, although they may also be of rodent origin. These proteins and the sequences that encode them are well known in the art. Such sequences of genes and proteins can be found on the Internet in databases provided by the National Center for Biotechnology Information, or other similar databases.

The presence of growth suppression activity of the ras proteins, or DNA or RNA that encode ras, is determined by one or more of a variety of assays. One group of such assays involve introduction of the gene or protein into cells in culture or in an animal and then measuring the effect of that gene or protein on growth of the cells. Growth suppression activity causes inhibition of growth. Another method is to introduce the gene or protein into cells and then measure the activity of ERK MAP kinase. This measurement can be made though the use of biochemical assays that are well known in the art.

The methods also provide for modifications to the ras gene or its encoded protein that result in stabilization or increased half-life of the protein in the body of the human or animal after administration thereto. It is important to note that any modifications to the wild-type ras gene or protein is within the scope of this invention, as long as the modified, shortened or partial protein retains some activity that can be said to be anti-proliferative, growth suppressing, anti-tumorigenic, anti-oncogenic, anti-malignant, and the like.

Introduction of the wild-type ras genes can be done using a variety of methodologies. Many of these methodologies are well known in the art of gene therapy. Genes can be introduced using a variety of methods such as transfection or infection of cells of a tumor or cancer with viral vectors encoding wild-type ras activity or causing stimulation of wild-type ras activity.

The invention provides for introduction of wild-type ras genes into cells that are present within a human or animal, such as for example, cells that comprise a tumor within a human or animal. There are a variety of methods for introducing genes into cells that are in a human or animal. One preferred method uses administration of recombinant viruses that contain a cloned wild-type ras gene into the human or animal such that the virus infects cells that comprise the tumor. Another method for introducing genes into cells that are in a human or animal involves administration of purified DNA that encodes wild-type ras directly into the human or animal. Such administration can be done by injection of the DNA. Such methodologies are commonly used in the vaccine field, specifically for administration of so-called “DNA vaccines.”

In order to introduce the polynucleotide sequences encoding wild-type ras activity into cells, the protein coding region of the polynucleotide sequences is normally attached to sequences that facilitate its transcription into mRNA as well as translation of the mRNA into wild-type ras. A strategy common in the art for doing this is to clone the polynucleotide sequence encoding the ras protein into a vector which contains sequences facilitating expression of a protein coding sequence cloned therein.

Expression vectors normally contain sequences that facilitate gene expression. An expression vehicle can comprise a transcriptional unit comprising an assembly of a protein encoding sequence and elements that regulate transcription and translation. Transcriptional regulatory elements generally include those elements that initiate transcription. Types of such elements include promoters and enhancers. Promoters may be constitutive, inducible or tissue specific. Transcriptional regulatory elements also include those that terminate transcription or provide the signal for processing of the 3′ end of an RNA (signals for polyadenylation). Translational regulatory sequences are normally part of the protein encoding sequences and include translational start codons and translational termination codons. There may be additional sequences that are part of the protein encoding region, such as those sequences that direct a protein to the cellular membrane, a signal sequence for example.

The ras sequences that are introduced into cells are preferably expressed at a high level (i.e., the introduced polynucleotide sequence produces a high quantity of ras protein within the cells) after introduction into the cells. Techniques for causing a high-level of expression of polynucleotide sequences introduced into cells are well known in the art. Such techniques frequently involve, but are not limited to, increasing the transcription of the polynucleotide sequence, once it has been introduced into cells. Such techniques frequently involve the use of transcriptional promoters that cause transcription of the introduced polynucleotide sequences to be initiated at a high rate. A variety of such promoters exist and are well known in the art. Frequently, such promoters are derived from viruses. Such promoters can result in efficient transcription of polynucleotide sequences in a variety of cell types. Such promoters can be constitutive (e.g., CMV enhancer/promoter from human cytomegalovirus) or inducible (e.g., MMTV enhancer/promoter from mouse mammary tumor virus). A variety of constitutive and inducible promoters and enhancers are known in the art. Other promoters that result in transcription of polynucleotide sequences in specific cell types, so-called “tissue-specific promoters,” can also be used. A variety of promoters that are expressed in specific tissues exist and are known in the art. For example, promoters whose expression is specific to neural, liver, epithelial and other cells exist and are well known in the art. Methods for making such DNA molecules (i.e., recombinant DNA methods) are well known to those skilled in the art.

In the art, vectors refer to nucleic acid molecules capable of mediating introduction of another nucleic acid or polynucleotide sequence to which it has been linked into a cell. One type of preferred vector is an episome, i.e., a nucleic acid capable of extrachromosomal replication. Other types of vectors become part of the genome of the cell into which they are introduced. Vectors capable of directing the expression of inserted DNA sequences are referred to as “expression vectors” and may include plasmids, viruses, or other types of molecules known in the art.

Typically, vectors contain one or more restriction endonuclease recognition sites which permit insertion of the ras polynucleotide sequence. The vector may further comprise a marker gene, such as for example, a dominant antibiotic resistance gene, which encode compounds that serve to identify and separate transformed cells from non-transformed cells.

One type of vector used in the present invention are viral vectors. Viral vectors are recombinant viruses which are generally based on various viral families comprising poxviruses, herpesviruses, adenoviruses, parvoviruses and retroviruses. Such recombinant viruses generally comprise an exogenous polynucleotide sequence (herein, the ras gene) under control of a promoter which is able to cause expression of the exogenous polynucleotide sequence in vector-infected host cells.

One type of viral vector is a defective adenovirus which has the exogenous polynucleotide sequence inserted into its genome. The term “defective adenovirus” refers to an adenovirus incapable of autonomously replicating in the target cell. Generally, the genome of the defective adenovirus lacks the sequences necessary for the replication of the virus in the infected cell. Such sequences are partially or, preferably, completely removed from the genome. To be able to infect target cells, the defective virus contains sufficient sequences from the original genome to permit encapsulation of the viral particles during in vitro preparation of the construct. Other sequences that the virus contains are any such sequences that are said to be genetically required “in cis.”

Another type of viral vector is a defective retrovirus which has the exogenous polynucleotide sequence inserted into its genome. Such recombinant retroviruses are well known in the art. Recombinant retroviruses for use in the present invention are preferably free of contaminating helper virus. Helper viruses are viruses that are not replication defective and sometimes arise during the packaging of the recombinant retrovirus.

Non-defective or replication competent viral vectors can also be used. Such vectors retain sequences necessary for replication of the virus.

Other types of vectors are plasmid vectors.

In one aspect, the present method comprises introduction of ras polynucleotide sequences, preferably contained within a vector, into specific cells so that the cells have increased levels of wild-type ras. Herein, such introduction or transfer of a DNA molecule or molecules, specifically a DNA molecule encoding one or more ras polynucleotide sequence, into a cell refers to any of a variety of methods known in the art to get DNA molecules into cells. One such method, whereby isolated DNA is introduced into cells, is know as transfection. Such transfection is commonly performed using various treatments of the cells or DNA which facilitate uptake of the DNA by the cell. For example, cells can be treated chemically to make them permeable to DNA. DNA can also be treated, for example by containing the DNA within liposomes that cells can internalize. Preferably, transfection is used to introduce plasmid DNA into cells.

As described above, ras polynucleotide sequences can also be introduced into cells using viruses. For example, polynucleotide sequences that are to be introduced into cells are cloned into viral genomes. Infection of cells with such viruses results in introduction of the viral genome into the cell. Since the cloned polynucleotide sequence is part of the viral genome, it is introduced into the cell along with the viral genome. Such viral “vectors” can have DNA or RNA genomes. Numerous such viral vectors are well known to those skilled in the art. Viral vectors that have cloned polynucleotide sequences, encoding ras proteins for example, cloned into their genomes are referred to as “recombinant” viruses. Transfer of DNA molecules using viruses is particularly useful for transferring polynucleotide sequences into particular cells or tissues of an animal. Such techniques are commonly known in the art as gene therapy.

Another method for introducing polynucleotide sequences into cells that are in a human or animal patient involves administration of purified DNA that contains polynucleotides encoding ras protein directly into the human or animal. Such administration can be performed by injection of the DNA or even transfection of DNA. Such methodologies are commonly used in the vaccine field, specifically for administration of so-called “DNA vaccines.”

Whatever methodology is used to administer the wild-type ras genes to humans or animal, such methodologies may comprise variations that result in the wild-type ras genes being preferentially introduced into tumor cells and less preferentially introduced into non-tumor cells. For example, techniques are known in the art that result in recombinant viruses specifically infecting certain cell types (e.g., tumor cell types) within a human or animal. For viruses, such “targeting” can be accomplished through manipulation of cellular receptors for the recombinant viruses and/or manipulation of viral ligands that recognize and bind to cellular receptors for the viruses. Such methodologies, as used to introduce wild-type ras genes into tumor cells in animals or humans, are within the purview of the present application.

There are methods known in the art of gene transfer and gene therapy for introducing exogenous polynucleotide sequences into specific cells and not into other cells. For example, techniques are known in the art that result in recombinant viruses specifically infecting certain cell types (e.g., tumor cell types) within a human or animal. For viruses, such “targeting” can be accomplished through manipulation of cellular receptors for the recombinant viruses and/or manipulation of viral ligands that recognize and bind to cellular receptors for the viruses. Such methodologies, as used to introduce ras polynucleotide sequence into tumor cells in animals or humans, are within the purview of the present application.

After ras polynucleotide sequences are introduced into cells, techniques are used to determine specifically the cells into which the polynucleotide sequences have been introduced and/or the specific cells that are expressing the introduced polynucleotide sequences. A variety of techniques to examine the presence of polynucleotide sequences and/or expression of polynucleotide sequences exist and are well known in the art. Some such techniques include Southern blotting, Northern blotting, polymerase chain reaction (PCR), Western blotting, RNase protection, radioiodide uptake assays, and others.

Wild-Type Ras Proteins as Therapeutic Agents

Another aspect of the present invention provides a method for inhibiting or suppressing growth of cells by introducing ras proteins into the cells. A variety of methods exist for introducing proteins into cells. There are a variety of methods known in the art for introducing proteins into cells. In one method, proteins are coupled or fused to short peptides that direct entry into cells. One such group of peptides are called protein transduction domain. Another method for introducing proteins into cells uses lipid carriers. For example, proteins that are associated with liposomes are able to enter cells when the liposomes enter or fuse with cells. Other methods of introducing proteins into cells are known. Microinjection and electroporation are two such methods. Other methods are known.

Such methods include, but are not limited to, “protein transduction” or “protein therapy” as described in publications by Nagahara et al. (Nagahara, et al., 1998, Nat Med, 4:1449-52.) and in publications from the laboratory of Dowdy (Nagahara, et al., 1998, Nat Med, 4:1449-52.; Schwarze, et al., 1999, Science, 285:1569-72.; Vocero-Akbani, et al., 2000, Methods Enzymol, 322:508-21; Ho, et al., 2001, Cancer Res, 61:474-7.; Vocero-Akbani, et al., 2001, Methods Enzymol, 332:36-49; Snyder and Dowdy, 2001, Curr Opin Mol Ther, 3:147-52.; Becker-Hapak, et al., 2001, Methods, 24:247-56.), publications which are incorporated herein by reference.

In one embodiment an eleven amino acid sequence, the “protein transduction domain” (PTD), from the human immunodeficiency virus TAT protein (Green and Loewenstein, 1988, Cell, 55:1179-88.; Frankel and Pabo, 1988, Cell, 55:1189-93.) is fused to the wild-type ras protein. The purified protein is then put in contact with the surface of cells and the cells take up the wild-type ras protein which functions to inhibit or suppress growth of that cell. In the case where it is desired to introduce the wild-type ras protein containing the fused PTD into cells comprising a tumor in a human or animal, the protein is administered to the human by a variety of methods. Preferably, the protein is administered by injection (e.g., intravenously) or by inhalation in an aerosol.

Wild-type ras proteins that contain the fused PTD are preferably made by fusing the DNA sequence encoding the wild-type ras gene with the DNA sequence encoding the PTD. The resulting ras-PTD fusion gene is preferably incorporated into a vector, for example a plasmid or viral vector, that facilitates introduction of the fusion gene into a organism and expression of the gene at high levels in the organism such that large amounts of the fusion protein are made therein. One such organism in which the vector containing the fusion gene can be expressed is a bacterium, preferably Escherichia coli. Other organisms are also commonly used by those skilled in the art. After the fusion protein is expressed at a high level in any of these organisms, the fusion protein is purified from the organism using protein purification techniques well known to those skilled in the art.

The wild-type ras proteins are administered to a human or other mammal, preferably in a pharmaceutical compositions. Suitable formulations for delivery are found in Remington's Pharmaceutical Sciences, 17th ed. (Mack Publishing Co., Philadelphia, Pa., 1985). These pharmaceutical compositions are suitable for use in a variety of drug delivery systems (Langer, Science 249:1527-1533, 1990).

Wild-type ras proteins in compositions are suitable for single administration or in a series of inoculations. The pharmaceutical compositions are intended for parenteral, topical or oral administration. Parenteral administration is preferably by intravenous, subcutaneous, intradermal, intraperitoneal or intramuscular administration. Parenteral administration may be preferentially directed to the patient's liver such as by catheterization to hepatic arteries or into a bile duct. For parenteral administration, the compositions can include ras proteins and a suitable sterile carrier such as water, aqueous buffer, 0.4% saline solution, 0.3% glycine, hyaluronic acid or emulsions of nontoxic nonionic surfactants as is well known in the art. The compositions may further include substances to approximate physiological conditions such a buffering agents and wetting agents such as NaCl, KCl, CaCl₂ sodium acetate and sodium lactate. It should be noted that, in addition to administration of ras protein in a pharmaceutically-acceptable composition, DNA or RNA encoding ras proteins may also be administered as a part of such a composition.

Solid compositions including ras proteins in conventional nontoxic solid carriers such as, for example, glucose, sucrose mannitol, sorbitol, lactose, starch, magnesium stearate, cellulose or cellulose derivatives, sodium carbonate and magnesium carbonate. For oral administration of solid compositions, the HCV-like particles preferably comprise 10% to 95%, and more preferably 25% to 75% of the composition.

A therapeutically or prophylactically effective dose of the ras protein composition is administered to the individual. The composition may be administered as a single dose, but more likely as a series of dosages over a period of days, weeks or even months. Herein, an effective therapeutic dose is a dose that inhibits growth of a tumor, or even causes tumor regression. Such a dose also will inhibit or prevent metastasis of a tumor. Herein, an effective prophylactic dose is a dose that prevents formation of a tumor.

Expression of Wild-Type ras on ERK MAP Kinase

Another aspect of the present invention provides a method for inhibiting the activity of ERK MAP kinase. Inhibition of ERK MAP kinase activity occurs after introduction into cells of a gene ericoding a wild-type ras protein or of a wild-type ras protein, as described above.

### EXAMPLES

Further details of the invention can be found in the following examples, which further define the scope of the invention. All references cited herein are expressly incorporated by reference in their entirety.

### Example 1

### Growth Inhibition of Tumor Cell Lines by Wild-Type K-ras Genes

A transfection study was conducted wherein wild-type K-ras inhibited growth of a transformed NIH/3T3 cell line named R16 (You, et al., 1989, Proc Natl Acad Sci USA, 86:3070-4.) and mouse lung tumor cell line named LM2 (McDoniels-Silvers, et al., 2001, Exp Lung Res, 27:297-318.). Both cell lines contained mutant K-ras alleles. R16 was derived from NIH/3T3 cells transformed with mouse lung tumor DNA containing a GC to AT transition at the second position of codon 12 in K-ras. R16 cells exhibited a high-level expression (around 10-fold higher than the wild-type K-ras allele) of mutant K-ras. LM2 is a metastatic tumor cell line established from urethane-induced lung tumors in the A/J mouse strain. LM2 contained an AT to GC transition at the second position of codon 61 in K-ras.

An expression vector containing wild-type K-ras was made from a full-length mouse cDNA for wild-type K-ras4B which was isolated by RT-PCR of total mRNA isolated from normal lungs of A/J mice using primers derived from the mouse cDNA sequences (George, et al., 1985, Embo J, 4:1199-203.). A wild-type K-ras4B expression construct was made by ligation of cDNAs into a pCR3.1 mammalian expression plasmid (Invitrogen, Carlsbad, Calif.). The expression construct was then purified using Maxi-tip 500 columns (QIAGEN, Santa Clarita, Calif.) and sequenced for confirmation prior to use in transfection experiments.

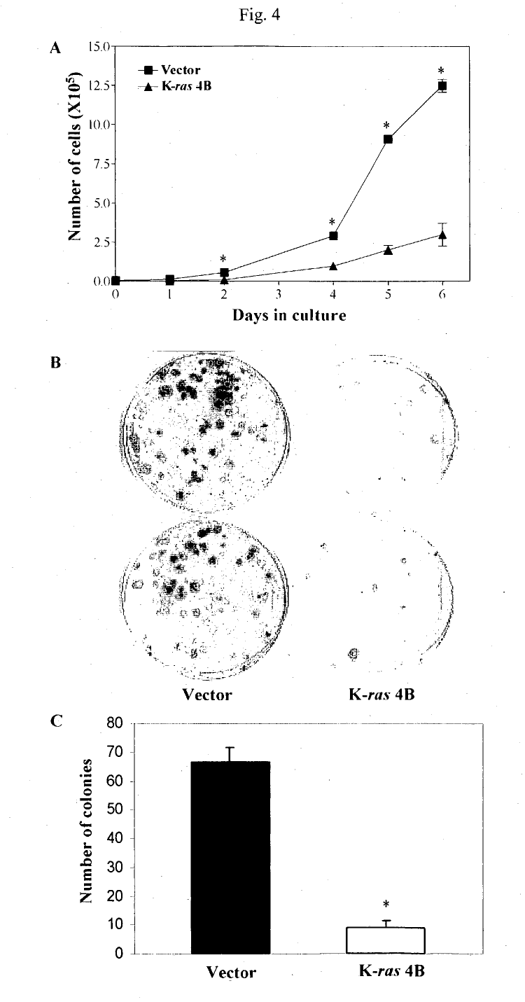

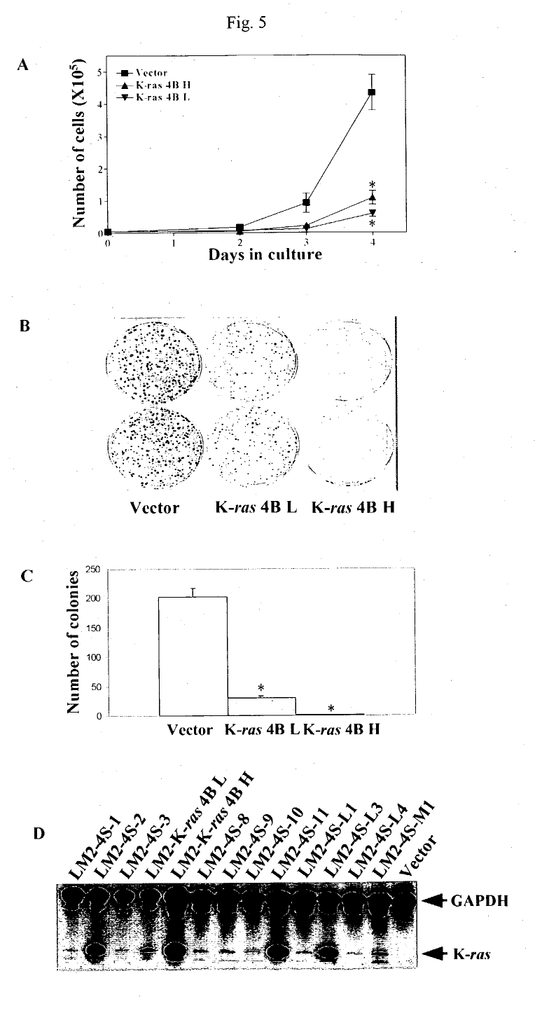

R16 and LM2 cells of the same passage were transfected under identical conditions with 1.5-2 μg of purified plasmid DNA of the following clones: pCR3.1 vector alone and wild-type K-ras4B using Lipofectamine (Life Technologies, Gaithersburg, Md.). Transfected cells were selected by selection in G418. The G418-resistant R16 cells were assayed directly and G418-resistant LM2 cells were subcloned into stable clones. These cells were used for analysis of growth rates and colony forming efficiencies. Growth rates were determined from these cells using 12-well dishes with two thousand cells seeded into each well. R16 cells were incubated in duplicate wells in Dulbecco's modified Eagle's medium (DMEM) plus 10% FBS and 50 μg/ml G418. LM2 cells were cultured in CMRL 1066 plus 10% FBS and 50 μg/ml G418. Cells were counted on days 1, 2, 4, 6, and 8 after plating (see FIGS. 4 and 5). For the colony-formation assay, 4 dishes were used, each 10-cm in diameter, for each subclone of transfected cells. One thousand cells were seeded into each dish and incubated in DMEM plus 10% FBS and 50 μg/ml G418 for 12 days. The cells were then fixed with 10% buffered formalin, stained with crystal violet, and visible colonies counted (>1.5 mm in diameter). Expression of the transfected wild-type K-ras4B was monitored in these subclones, and in selected colonies from the colony formation assay, by RT-PCR utilizing unique sequences carried by the transfected clone. Specifically, cDNA was generated from RNA isolated from G418-resistant cells that had been transfected with wild-type K-ras4B. PCR was then performed on these cDNAs using a primer specific for a vector sequence located behind transcription start position (5′TAATACGACTCACTATAGGG3′) and a K-ras4B intragenic coding sequence (5′CTCTATCGTAGGGTCGTAC3′).

As shown in FIG. 4a, transfection of wild-type K-ras into R16 cells significantly inhibited cell growth (80% inhibition by day 6). While more than 65 colonies grew in empty vector control cells, very few colonies (less than 10) arose from the wild-type K-ras-transfected NIH/3T3 cells were observed (FIGS. 4b & c). Transfection of the wild-type K-ras gene into LM2 significantly inhibited cell growth (90% inhibition by day 4) in one of the selected clones of the G418-resistant cells (LM2-K-ras4B H) (FIG. 5a). LM2-K-ras4B H expressed a relatively high level of transfected wild-type K-ras (FIG. 5d). While more than 200 colonies grew in empty vector control cells, very few colonies (less than 10) were seen in LM2-K-ras4B H cells (FIGS. 5b & c). An average of 30 colonies from LM2 cells expressed ˜3-fold lower level (designated as LM2-K-ras4B L) of the transfected wild-type K-ras (FIG. 5d), indicating that the inhibitory effect of wild-type K-ras is dose-dependent (FIGS. 5b-d).

It was tested whether the inhibitory effect of transfected wild-type K-Ras could be explained by a decrease in protein expression of oncogenic K-Ras. LM2 cells contain a CAA to CGA mutation in codon 61 of K-ras. There are no antibodies available that recognize this mutant K-Ras; therefore, an alternative method was used. GTP-bound but not GDP-bound Ras can specifically associate with the Ras binding domain (RBD) of c-Raf. Bacterially expressed GST-RBD was used as an affinity matrix to isolate the GTP-bound, oncogenic form of K-Ras in the LM2 cell lines.

LM2 cells were lysed in mild lysis buffer (10 mM Tris-HCl pH-7.5, 100 mM NaCl, 1% NP-40, 5 mM MgCl₂, 0.2 mM PMSF, 2 μg/ml leupeptin, 5 μg/ml aprotinin, 1 mM benzamidine) and cleared by centrifugation. Protein concentrations were determined by Bradford Assay (BioRad) and samples equalized prior to incubation with either 40 μg GST-RBD of Raf (Herrmann, et al., 1995, J Biol Chem, 270:2901-5.), pre-coupled to glutatione agarose or glutathione agarose alone. After 2 hrs of mixing, the beads were washed and bound proteins eluted with SDS sample treatment buffer. Bound K-Ras protein was visualized by Western blot with α-KRas F234 antibody (Santa Cruz Biotechnology).

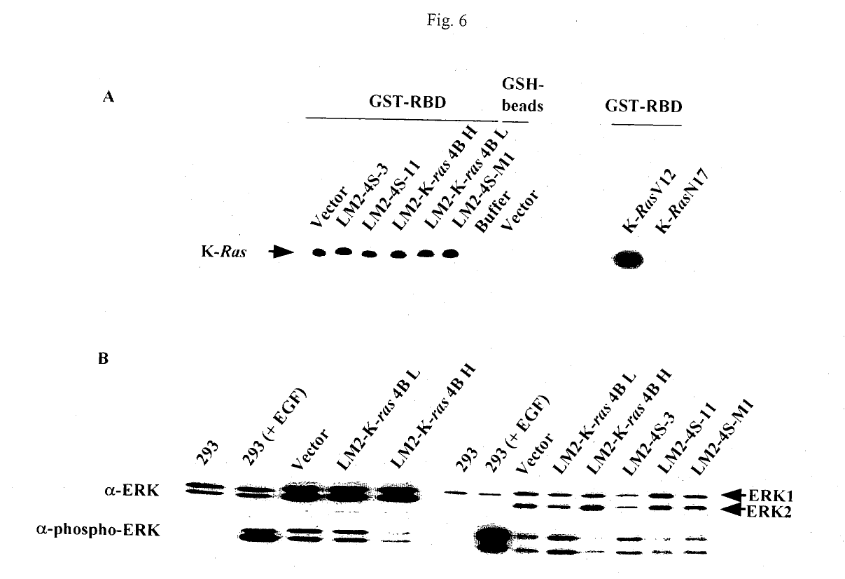

As shown in FIG. 6a, equivalent amounts of oncogenic K-Ras were isolated in all the cell lines tested that had varying degrees of expression of transfected wild-type K-Ras. LM2-4S-11 and LM2-K-ras4B H expressed relatively high levels of transfected wild-type K-Ras while LM2-4S-3, LM2-K-ras4B L, and LM2-4S-M1 had a relatively low level of transfected wild-type K-Ras. As a positive control, 293 cells were transfected with either K-RasV12 (pCDNA3-K-RasV12; GTP-bound) or K-RasN17 (pCDNA3-K-RasN17; GDP-bound) and only K-RasV12 was associated with GST-RBD (FIG. 6a). The data show that the expression of oncogenic K-Ras is not affected by overexpression of the wild-type form.

### Example 2

### Wild-Type K-Ras Inhibited ERK Activity

These studies determined that expression of wild-type K-Ras affects ERK activity. Activation of MAP kinase is known to play an important role in cellular transformation. ERK activity was determined by immunblotting with anti-phospho-ERK antibody. ERK activity is directly regulated by phosphorylation of the activation loop, which is recognized by anti-phospho-ERK. The ERK kinase activity directly correlates with signals detected using an anti-phospho-ERK immunoblot.

As described in Example 1, LM2-K-ras4B H and LM2-4S-11 cell lines were used in these studies. HEK293 cells were also used as controls. For analyzing ERK activity, HEK293 and LM2 cells were cultured in DMEM and CMLR1066 media supplemented with 10% FBS, respectively. Fifty ng/ml of EGF was used to stimulate ERK activity for 10 min. HEK cells without stimulation with EGF were included as a negative control and HEK293 cells stimulated with EGF were used as a positive control. As described in Example 1 above, cells were lysed directly in SDS sample treatment buffer and analyzed by Western blotting with anti-ERK antibody or anti-phospho-ERK antibody (Sigma Chemical Co. St. Louis, Mo.).

As shown in FIG. 6b, an inverse correlation between the level of wild-type K-Ras expression and ERK activity was observed. LM2-K-ras4B H and LM2-4S-11 cell lines had a relatively high level of K-Ras expression (FIG. 5d) and displayed a significantly lower ERK activity than vector control cells (FIG. 6b). In contrast, LM2-K-ras4B L, LM2-4S-3, and LM2-4S-M1 cells had a relatively lower level K-Ras expression (FIG. 5d) and showed a relatively normal ERK activity (FIG. 6b).

### Example 3

### Transfected Wild-Type K-ras Inhibited in vivo Tumor Growth of Transfected Cell Lines

This study showed that wild-type K-ras inhibited tumor development by R16 and LM2 cells in nude mice. Wild-type K-ras4B or an empty vector were transfected into R16 and LM2 cells as described above in Example 1. Athymic mice (4-6 week-old) were obtained from the Jackson Laboratory (Bar Harbor, Me.). Approximately 1 million cells in log phase growth were injected subcutaneously into each flank of nude mice; five animals were used per subclone. The health of the animals was monitored and the size of tumors checked daily and recorded tumor latency (time of appearance) and size (mm³). Mice were euthanized at two to four weeks after cell injection and measured tumor weights. The expression of the wild-type K-ras clone in all nude mouse tumors was confirmed by RT-PCR.

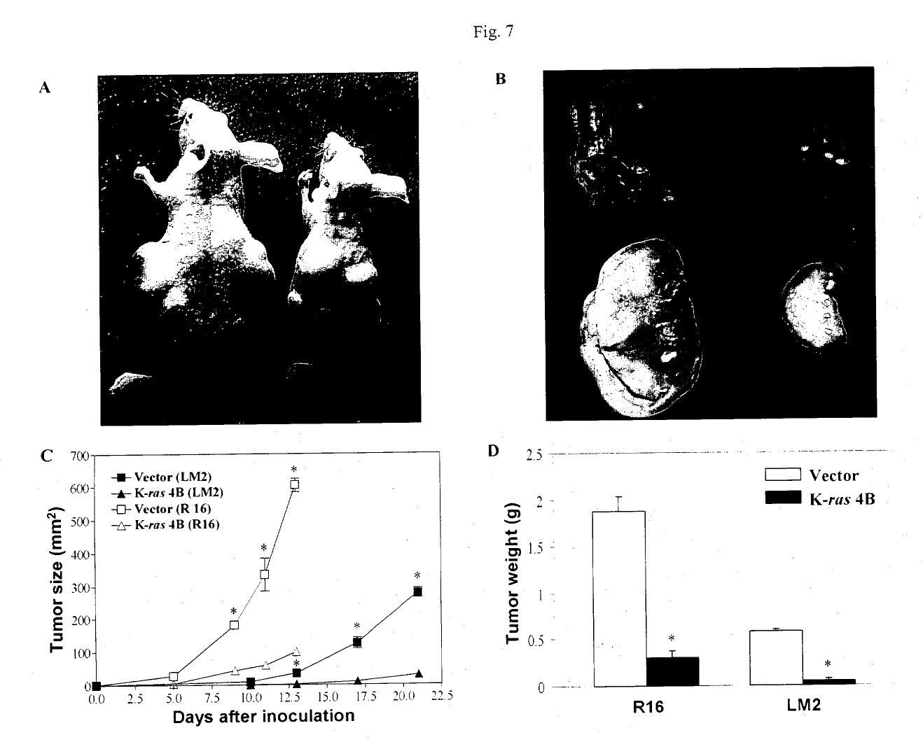

As shown in FIGS. 7a-d, large tumors developed in all five mice injected with vector control cells, whereas only small tumors were observed in the five mice injected with R 16 cells and LM2 cells carrying wild-type K-ras. Tumors in the wild-type K-ras-transfected group were significantly smaller (˜0.3 g and ˜0.05 g) (P<0.0001) compared to tumors in vector control mice (˜1.9 g and ˜0.6 g) (FIG. 7d). These results indicated that wild-type K-ras inhibits the tumorigenic potential of R16 and LM2 cells.

### Example 4

### Administration of Wild-Type K-ras Genes to Humans using a Viral Vector

A recombinant adenovirus is constructed by recombining a wild-type K-ras gene into the adenoviral genome. Virus is grown and purified using standard methods. To test the ability of the recombinant K-ras adenovirus to express the K-ras gene, cultured cells are infected, protein extracts are made from the infected cells and the extracts are analyzed for the presence of K-ras protein by immunoblotting using an antibody specific for the K-ras protein. Recombinant adenoviruses that express K-ras are administered to humans that have tumors. Growth of the tumors in the humans to which the recombinant adenovirus has been administered is monitored thereafter.

### Example 5

### Administration of Wild-Type K-ras Protein to Humans using Protein Transduction

A bacterial plasmid expression vector is constructed that contains a wild-type K-ras gene with a nucleotide sequence encoding an 11-amino acid protein transduction domain (PTD) from the human immunodeficiency virus TAT gene fused to the 5′ end of the wild-type K-ras gene. The plasmid is transformed into Escherichia coli which produce the wild-type K-ras protein with the 11-amino acid PTD fused to the amino-terminal end of the protein (K-ras:PTD fusion protein). The K-ras:PTD fusion protein is purified from the transformed Escherichia coli using standard protein purification methodologies. The purified K-ras:PTD fusion protein is administered to humans that have tumors. Administration is either by intravenous injection or by inhalation of an aerosol. The K-ras:PTD fusion protein that is administered to humans is contained within a pharmaceutical preparation that is acceptable for the particular route of administration.

### Example 6

### Diagnosis of Cancers Caused by Activated ras and Loss of Wild-Type ras

A tissue sample from a tumor in a human is obtained by biopsy. The cells comprising the tissue sample are lysed and DNA is isolated from the cells using standard methods. The isolated DNA is subjected to PCR using primers that amplify regions of K-ras genomic sequences that encode the K-ras protein. The amplified PCR products and then sequenced. The obtained DNA sequences are analyzed to determine if known activating mutations are present and also to determine whether additional mutations in K-ras are present that result in elimination of growth suppression activity of wild-type K-ras protein.

### Example 7

### Characterization of Cancers Related to Loss of Wild-Type ras

A total of 22 adenocarcinomas and 8 large cell carcinomas were genotyped for loss of heterozygosity on chromosome 12. These tumors and their paired normal tissues were obtained from the Cooperative Human Tissue Network of The Ohio State University Department of Pathology (Columbus, Ohio) and the University of Cincinnati (Cincinnati, Ohio). A pathologist classified all tumors histopathologically. High molecular weight DNA was isolated from both tumor and normal tissues of each case according to published protocols Blin, N. & Stafford, D. W. (1976). Nucleic Acids Res, 3, 2303-8.) Allelic losses were assayed by PCR using eight polymorphic microsatellite markers on chromosome 12p: D12S89, D12S358, D12S310, D12S1606, D12S1596, G60541, D12S1617 and D12S1592 (Research Genetics, Inc.). All markers were scored on an 8% denaturing polyacrylamide gel. The gel was dried and exposed to X-ray film over night, and LOH was scored visually. Only those samples in which a 40% or more difference were scored as an allelic loss. Using PCR-direct sequencing analysis, Kras2 gene codon 12 mutations were determined in all lung adenocarcinoma and large cell carcinoma DNAs. PCR amplification of Kras2 exon 1 from lung tumors was carried out as described ou, M., Wang, Y., Stoner, G., You, L., Maronpot, R., Reynolds, S. H. & Anderson, M. (1992). Proc Natl Acad Sci USA, 89, 5804-8. The sequences of PCR primers for Kras2 exon I were Kras2-1F: 5′-TTTTTATTATAAGGCCTGCT-3′, SEQ ID NO. ______ and Kras2-1R: 5′-GTCCACAAAATGATTCTGAA-3′, SEQ ID NO. ______. The 114 bp PCR products were eluted using QIAquick gel extraction kit (Qiagen, Valencia, Calif.). Codon 12 mutations were detected using an ABI PRISM 3700 DNA analyzer (Perkin-Elmer/Applied Biosystems).

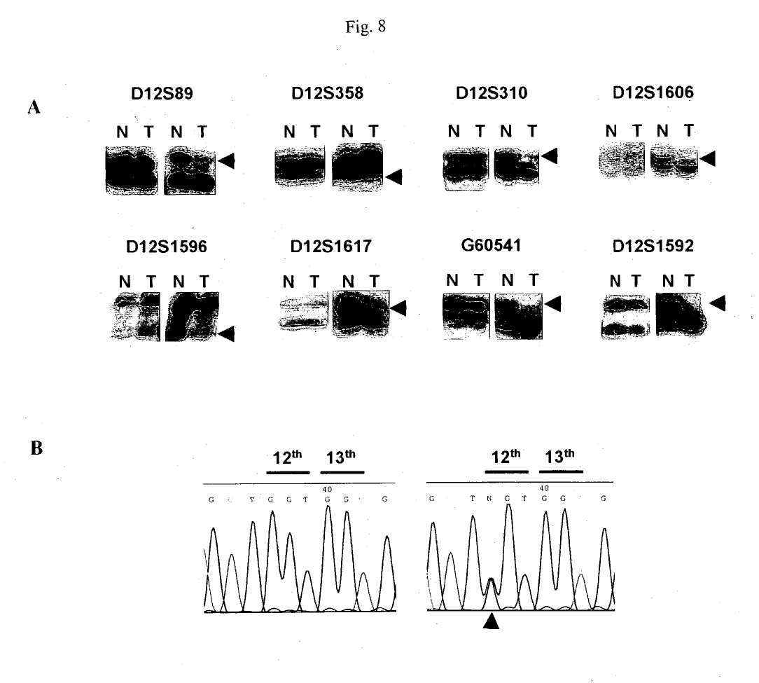

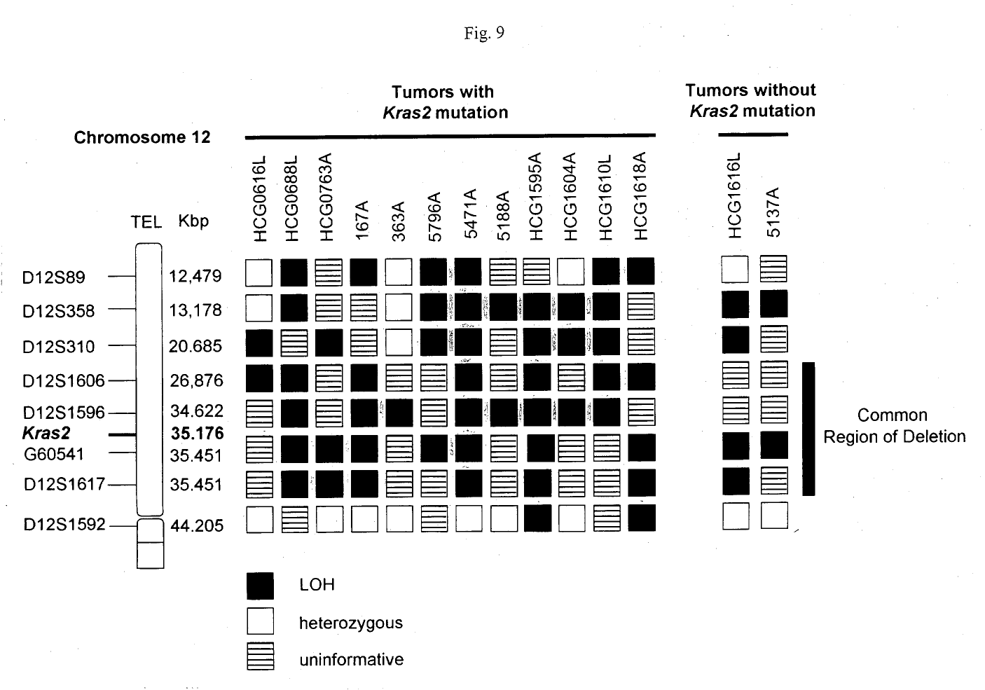

In total, eight microsatellite markers were analyzed with focus on the Kras2 region. Allelotype data for individual markers is summarized in Table 3. Eleven of 22 (50.0%) adenocarcinomas and 4 of 8 (50.0%) large cell carcinomas were found to have allelic loss at least on one of the markers used. FIG. 8a shows representative results of the allelotype analysis of chromosome 12p at eight microsatellite markers. For Kras2 mutation analysis, our data showed 9 of 22 (40.9%) of adenocarcinomas and 3 of 8 (37.5%) of large cell carcinomas contained a Kras2 mutation at codon 12 (GGT→TGT, CGT, GAT, GCT, and GTT transition; FIG. 8b, Table 3). When LOH analysis and Kras2 mutation data were combined, the majority of the allelic loss on chromosome 12p observed in adenocarcinomas and large cell carcinomas corresponded with an activated Kras2 mutation. Approximately 82% (9 of 11) of adenocarcinomas and 75% (3 of 4) of large cell carcinomas exhibited allelic loss of chromosome 12p while each retained the activated Kras2 allele. Twelve tumors with Kras2 mutations and with LOH at one or more loci on 12p are shown in FIG. 9. Of these, tumors HCG0688L, 5796A, HCG1595A, HCG1610L and HCG1618A displayed losses at all of the informative markers analyzed, whereas tumor 363A lost only the D12S1596 marker while retaining heterozygosity at the nearest informative markers, D12S310 and D12S1592. Tumors 167A and 5471A also displayed terminal deletion for marker D12S1617/G60541, where Kras2 is located, with retention of heterozygosity at the nearest informative marker, D12S1592 (FIG. 9). The LOH data localized a minimal region of LOH to markers D12S1606 and D12S1617/G60541. The physical genomic map has placed the Kras2 gene at a position that is flanked by these two markers in a ˜800 kb region. The results indicate that the wild type Kras2 allele is lost in human lung adenocarcinomas and large cell carcinomas,

TALBE 3
LOH on chromosome 12 in non small cell lung cancer

Frequency of LOH on chromosome 12

| | Adenocarcinoma | Large cell carcinoma |
|---|---|---|
| Marker | LOH/I^{a} (%) | LOH/I (%) |
| 1.D12S89 | 4/11 (36.4%) | 2/5 (40.0%) |
| D12S358 | 6/11 (54.5%) | 3/6 (50.0%) |
| D12S310 | 5/11 (45.5%) | 3/6 (50.0%) |
| D12S1606 | 4/8 (50.0%) | 3/4 (75.0%) |
| D12S1596 | 6/10 (60.0%) | 2/3 (66.7%) |
| G60541 | 7/16 (43.8%) | 2/3 (66.7%) |
| D12S1617 | 5/13 (38.5%) | 2/4 (50.0) |
| D12S1592 | 2/12 (16.7%) | 0/3 (0.0%) |
| Combined | 10/22 (45.5%) | 4/8 (50.0%) |

I^{a}, number of tumors that were informative for the marker tested.

What is claimed is:

1. A method of inhibiting proliferation of a cell, comprising:
increasing intracellular levels of one or more wild-type ras proteins in the cell.

2. The method of claim 1 wherein the intracellular levels of wild-type ras protein are increased in the cell by
a) introducing a nucleic acid that comprises a coding region for a wild-type ras protein into the cell, and
b) expressing the wild-type ras protein in the cell.

3. The method of claim 1 wherein the cell is a tumor cell in a human or other mammal.

4. The method of claim 1 wherein said nucleic acid is a DNA comprising a promoter sequence that causes a high level of transcription of the wild-type ras protein encoding region.

5. The method of claim 1 wherein said nucleic acid is part of a viral genome.

6. The method of claim 1 wherein the wild-type ras protein is selected from the group consisting of wild-type K-ras protein, wild-type N-ras protein, and wild-type H-ras protein, or combinations thereof.

7. The method of claim 1 wherein intracellular levels of wild-type ras proteins are increased in said cell by introducing one or more wild-type ras protein into the cell.

8. The method of claim 7 wherein the cell is a tumor cell in a human or other animal.

9. The method of claim 7 wherein the wild-type ras protein additionally comprises a protein transduction domain.

10. The method of claim 9 wherein the protein transduction domain comprises an 11 amino acid sequence from the human immunodeficiency virus TAT protein.

11. A method of decreasing the activity of ERK MAP kinase in a cell comprising increasing the levels of wild-type ras protein in the cell.

12. A method of evaluating a tumor in a mammalian subject, comprising:
providing cells from the tumor; and
assaying for a loss of function mutation in at least one of the endogenous ras alleles in the genome of the cells.

13. The method of claim 12 further comprising assaying for an activating mutation in one of the endogenous ras allelles in the genome of said cells.

14. The method of claim 12 wherein the loss of function mutation results in production of a mutated ras protein having decreased cell proliferation activity.

15. The method of claim 12 wherein the loss of function mutation results in reduced levels of ras protein in the tumor cells as compared to the levels of ras protein in normal cells.

16. The method of claim 12 wherein the loss of function mutation results in loss of all or part of a wild-type ras allele.

17. The method of claim 13 wherein the presence of a loss of function mutation in one ras allele and an activating mutation in another ras allele is indicative of a poor prognosis.

18. The method of claim 12 wherein the loss of function mutation is in the first, second, or third exon of the ras allele.

19. The method of claim 12 wherein the loss of function mutation is assayed by determining the intracellular levels of one or more wild-type ras proteins in the cell.

20. The method of claim 12 wherein the loss of function mutation is assayed by determining the intracellular levels of mRNA encoding one or more wild-type ras proteins.

21. The method of claim 12 wherein the loss of function mutation is assayed by detecting the presence or absence of one or more wild-type ras allelles in the genome of the cell.

22. The method of claim 12 wherein the loss of function mutation is assayed by determining the cell proliferation activity of one or more wild-type ras proteins in the cell.

23. A method for preventing or treating tumors or cancers in a mammalian subject, comprising:
administrating a wild-type ras protein or nucleic acids that encode and express a wild-type ras protein to the subject.

24. The method of claim 23 wherein a wild-type ras protein is injected into a tumor in the subject.

25. The method of claim 23 wherein a nucleic acid encoding a wild-type protein is injected into the subject.

26. A method for prognosis of a cancer in a human subject comprising assaying for a loss of function mutation in one or more ras alleles in cancer cells obtained from the subject.

27. A method of inducing lung tumor formation in a heterozygous ras-knockout mouse, comprising injecting a chemical carcinogen into said mouse.
